(19) United States
(12) Patent Application Publication
(10) Pub. No.: US 2025/0283152 A1
(43) Pub. Date: Sep. 11, 2025

(54) NUCLEIC ACID AMPLIFICATION USING PROMOTER PRIMERS

(71) Applicant: Sherlock Biosciences, Inc., Watertown, MA (US)

(72) Inventors: Jonathan S. Gootenberg, Brighton, MA (US); Omar O. Abudayyeh, Brighton, MA (US); Xiang Li, Malden, MA (US); Mary Wilson, Brighton, MA (US); William Jeremy Blake, Winchester, MA (US)

(21) Appl. No.: 18/276,110

(22) PCT Filed: Feb. 8, 2022
(86) PCT No.: PCT/US2022/015553

§ 371 Date: Aug. 7, 2023

Related U.S. Application Data
(60) Provisional application No. 63/147,569, filed on Feb. 9, 2021.

(51) Int. Cl. C12Q 1/6823; C12Q 1/6844

(52) U.S. Cl. CPC C12Q 1/6823; C12Q 1/6844

(57) ABSTRACT

The present disclosure provides methods and compositions for nucleic acid amplification.

### CROSS REFERENCE TO RELATED APPLICATIONS

This application claims priority to U.S. Provisional Patent Application No. 63/147,569, filed Feb. 9, 2021, the entire contents of which are hereby incorporated by reference.

### BACKGROUND

Templated nucleic acid synthesis is an important step in many applications. Improvement of templated nucleic acid synthesis technologies, for example that may lead to increased speed, efficiency, and/or extent or quality of product generation are useful.

### SUMMARY

The present disclosure provides insights and technologies that can achieve improvement of nucleic acid production (e.g., amplification).

In some embodiments, the present disclosure provides a method comprising: contacting a templated nucleic acid synthesis target with: a plurality of primers, wherein: (1) the plurality includes at least one set of forward and reverse primers, each of which includes a templated nucleic acid synthesis target hybridization element selected so that the primers of the pair bind to opposite strands of a templated nucleic acid synthesis target, flanking a target sequence of interest; and (2) the plurality of primers furthermore includes at least two T7 promoter sequence elements; and (b) amplification reagents; incubating the templated nucleic acid synthesis target, plurality of primers; and amplification reagents so an amplified nucleic acid comprising the target sequence of interest and the at least two T7 promoter sequence elements is generated; contacting the amplified nucleic acid comprising the target sequence of interest with a CRISPR-Cas detection composition; and detecting the amplified nucleic acid.

In some embodiments, the Cas detection composition comprises: (i) a guide polynucleotide capable of binding the target sequence of interest; (ii) a labeled nucleic acid reporter construct; and (iii) at least one Cas protein. In some embodiments, the method comprises transcribing a copied and/or amplified templated nucleic acid synthesis target using any primer inserted promoter. In some embodiments, the Cas protein is a Cas13. In some embodiments, the Cas protein is a Cas12.

In some embodiments, the present disclosure provides a composition comprising: (a) a plurality of primers, wherein the plurality includes at least one set of forward and reverse primers, each of which includes a templated nucleic acid synthesis target hybridization element selected so that at least one forward primer and at least one reverse primer bind to opposite strands of a template nucleic acid synthesis target, flanking a target sequence of interest; wherein the plurality of primers furthermore includes at least two T7 promoter sequence elements and each templated nucleic acid synthesis target hybridization element is at least 80% complementary to its hybridization site in the target nucleic acid; (b) amplification reagents; and (c) a CRISPR-Cas detection composition.

In some embodiments, the present disclosure provides an amplified nucleic acid product comprising at least two T7 promoters and a target sequence of interest.

In some embodiments, the present disclosure provides a method of detecting a target sequence in a nucleic acid sample with an RNA-dependent CRISPR/Cas enzyme, the improvement that comprises: amplifying the target nucleic acid sequence with one or more primers that each comprise a T7 promoter element and a hybridization element.

In some embodiments, the present disclosure provides a system comprising: (i) an amplified nucleic acid comprising: a) a target sequence of interest; and b) a plurality of promoter elements; (ii) an RNA polymerase that polymerizes from promoter(s) found in amplified nucleic acid; (iii) a guide polynucleotide capable of binding the target sequence of interest; (iv) a labeled nucleic acid reporter construct; and (v) an RNA-dependent CRISPR/Cas. In some embodiments, the RNA-dependent CRISPR/Cas is a thermostable Cas. In some embodiments, the labeled nucleic acid reporter construct is fluorescently labeled.

### BRIEF DESCRIPTION OF THE DRAWING

FIG. 1 shows an exemplary system for target nucleic acid amplification and/or detection (e.g., using SHERLOCK detection technologies).

FIG. 2 shows an exemplary LAMP reaction.

FIG. 3 shows an exemplary LAMP reaction further comprise use of loop primers.

FIG. 4 shows a cartoon diagram comparing a conventional LAMP reaction with an exemplary LAMP reaction utilizing primers including multiple promoters (e.g., multiple promoters within a single LAMP primer and/or at least one promoter on each of at least two LAMP primers).

FIG. 5 shows exemplary LAMP primer locations, relative to one another, on a target nucleic acid (top) and exemplary design of LAMP primers including a promoter (bottom).

FIG. 6 demonstrates exemplary SHERLOCK assay performance coupled with LAMP using primers including a T7 promoter element. The heat-map color indicates the Limit of Detection (LOD) of each assay (log(aM)) using certain primer designs.

FIG. 7 demonstrates an exemplary SHERLOCK assay performance coupled with LAMP using primers including a T7 promoter element. The heat-map color indicates the Limit of Detection (LOD) of each assay (log(aM)) using certain primer designs.

FIG. 8 shows exemplary LAMP reaction speeds from LAMP reactions using primers including a T7 promoter element at various locations within primer. Four letter design designations represent the T7 promoter design on a FIP, BIP, LF, or LB primer, respectively. N means “no T7 promoter,” F means “forward T7 promoter”, and R means “reverse T7 promoter” (i.e., RFNN means FIP primer has reverse T7 promoter, BIP primer has forward T7 promoter, LF and LB have no T7 promoter). The control utilized the same set of primers, none of which included a T7 promoter sequence (e.g., NNNN). Old designs mean T7 promoters were all located on FIP/BIP primers, while New designs mean T7 promoters were all located on LF/LB primers.

FIG. 9 shows exemplary LAMP reaction speeds from LAMP reactions using primers including a T7 promoter element on inner verses loop primers.

FIG. 10 demonstrates T7 promoter elements may not be required in LAMP primers to subsequently achieve robust transcription of amplified target nucleic acid.

NTC=No transcript control. Cp/μL=copies of genomic viral RNA per μL template added to the reaction.

FIG. 11 demonstrates T7 polymerase and rNTPs are required for Cas13a-based detection.

### DEFINITIONS

Agent: In general, the term “agent”, as used herein, is used to refer to an entity (e.g., for example, a lipid, metal, nucleic acid, polypeptide, polysaccharide, small molecule, etc, or complex, combination, mixture or system [e.g., cell, tissue, organism] thereof), or phenomenon (e.g., heat, electric current or field, magnetic force or field, etc). In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Amino acid: in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Approximately or About: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

Biological Sample: As used herein, the term “biological sample” typically refers to a sample obtained or derived from a biological source (e.g., a tissue or organism or cell culture) of interest, as described herein. In some embodiments, a source of interest is or comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Cellular lysate: As used herein, the term “cellular lysate” or “cell lysate” refers to a fluid containing contents of one or more disrupted cells (i.e., cells whose membrane has been disrupted). In some embodiments, a cellular lysate includes both hydrophilic and hydrophobic cellular components. In some embodiments, a cellular lysate includes predominantly hydrophilic components; in some embodiments, a cellular lysate includes predominantly hydrophobic components. In some embodiments, a cellular lysate is a lysate of one or more cells selected from the group consisting of plant cells, microbial (e.g., bacterial or fungal) cells, animal cells (e.g., mammalian cells), human cells, and combinations thereof. In some embodiments, a cellular lysate is a lysate of one or more abnormal cells, such as cancer cells. In some embodiments, a cellular lysate is a crude lysate in that little or no purification is performed after disruption of the cells; in some embodiments, such a lysate is referred to as a “primary” lysate. In some embodiments, one or more isolation or purification steps is performed on a primary lysate; however, the term “lysate” refers to a preparation that includes multiple cellular components and not to pure preparations of any individual component.

Composition: Those skilled in the art will appreciate that the term “composition”, as used herein, may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. . . . For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Designed: As used herein, the term “designed” refers to an agent (i) whose structure is or was selected by the hand of man; (ii) that is produced by a process requiring the hand of man; and/or (iii) that is distinct from natural substances and other known agents.

Detectable entity: The term “detectable entity” as used herein refers to any element, molecule, functional group, compound, fragment or moiety that is detectable. In some embodiments, a detectable entity is provided or utilized alone. In some embodiments, a detectable entity is provided and/or utilized in association with (e.g., joined to) another agent. Examples of detectable entities include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Gel: As used herein, the term “gel” refers to viscoelastic materials whose rheological properties distinguish them from solutions, solids, etc. In some embodiments, a composition is considered to be a gel if its storage modulus (G′) is larger than its modulus (G″). In some embodiments, a composition is considered to be a gel if there are chemical or physical cross-linked networks in solution, which is distinguished from entangled molecules in viscous solution.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

In vitro: The term “in vitro” as used herein refers to events that occur in an artificial environment, e.g., in a test tube or reaction vessel, in cell culture, etc., rather than within a multi-cellular organism.

Isolated: as used herein, refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the systems and/or methods provided herein. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Specific: The term “specific”, when used herein with reference to an agent having an activity, is understood by those skilled in the art to mean that the agent discriminates between potential target entities or states. For example, an in some embodiments, an agent is said to bind “specifically” to its target if it binds preferentially with that target in the presence of one or more competing alternative targets. In many embodiments, specific interaction is dependent upon the presence of a particular structural feature of the target entity (e.g., an epitope, a cleft, a binding site). It is to be understood that specificity need not be absolute. In some embodiments, specificity may be evaluated relative to that of the binding agent for one or more other potential target entities (e.g., competitors). In some embodiments, specificity is evaluated relative to that of a reference specific binding agent. In some embodiments specificity is evaluated relative to that of a reference non-specific binding agent. In some embodiments, the agent or entity does not detectably bind to the competing alternative target under conditions of binding to its target entity. In some embodiments, binding agent binds with higher on-rate, lower off-rate, increased affinity, decreased dissociation, and/or increased stability to its target entity as compared with the competing alternative target(s).

Specificity: As is known in the art, “specificity” is a measure of the ability of a particular ligand to distinguish its binding partner from other potential binding partners.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments a human subject is an adult, adolescent, or pediatric subject. In some embodiments, a subject is suffering from a disease, disorder or condition, e.g., a disease, disorder or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Suffering from: An individual who is “suffering from” a disease, disorder, and/or condition displays one or more symptoms of a disease, disorder, and/or condition and/or has been diagnosed with the disease, disorder, or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

### DETAILED DESCRIPTION OF CERTAIN EMBODIMENTS

Templated nucleic acid synthesis (e.g., copying and/or amplification) and/or nucleic acid detection are critical tools in biomedical research and clinical medicine, including for use in a variety of diagnostic technologies. Established templated nucleic acid synthesis techniques include, but are not limited to, polymerase chain reaction (PCR), self-sustained sequence replication (S3R), strand displacement amplification (SDA), and loop-mediated isothermal amplification (LAMP). Embodiments disclosed herein provide improved technologies of templated nucleic acid synthesis under isothermal conditions (e.g., improved LAMP technologies). Traditional LAMP technologies amplify nucleic acid under isothermal conditions using two or more sets of primers and a polymerase with high strand displacement activity. In fact, typically a plurality of primers are utilized and in some embodiments, one or more primers includes a single promoter (e.g., a T7 promoter).

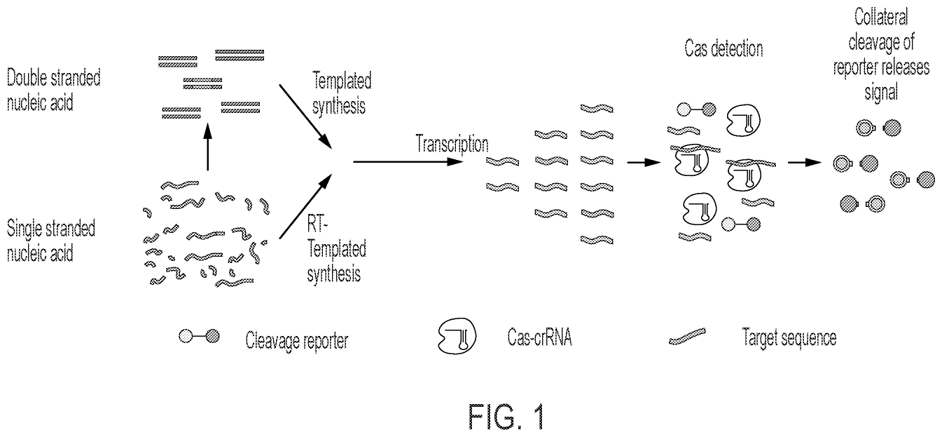

In some embodiments, technologies disclosed herein provide and/or otherwise relate to a system for templated nucleic acid synthesis and/or detection, an exemplary overview for which is shown in FIG. 1. In some embodiments, a disclosed system can comprise reagents for converting a nucleic acid to a double-stranded nucleic acid prior to templated nucleic acid synthesis. In some embodiments, the present disclosure documents transcription of LAMP product and subsequent detection. In some embodiments, the present disclosure documents that use of multiple promoters (e.g., multiple promoters within a single LAMP primer and/or at least one promoter on each of at least two LAMP primers) can achieve increased generation of transcript (e.g., relative to other methods of templated nucleic acid synthesis or LAMP without multiple promoters). In some embodiments, generation of increased transcripts can, among other things, decrease the duration of time required for detecting a nucleic acid. In some embodiments, the present disclosure documents that, even without the use of any primer in any promoter, LAMP product can be successfully transcribed and/or detected. In some embodiments, a detection method comprises a CRISPR-Cas based detection method (e.g., CRISPR-SHERLOCK). In some embodiments, a disclosed system for templated nucleic acid synthesis and/or detection of a nucleic acid occurs in a single reaction vessel (“one-pot” embodiment).

### Templated Nucleic Acid Synthesis

Templated nucleic acid synthesis permits copying and/or amplification of nucleic acids of interest (e.g., templated nucleic acid synthesis targets of interest) and is a central tool in biomedical research and clinical medicine, including specifically for a variety of diagnostic technologies. Established technologies for templated nucleic acid synthesis methods include, but are not limited to, polymerase chain reaction (PCR), primer extension, self-sustained sequence replication (3SR), strand displacement amplification (SDA), and loop-mediated isothermal amplification (LAMP).

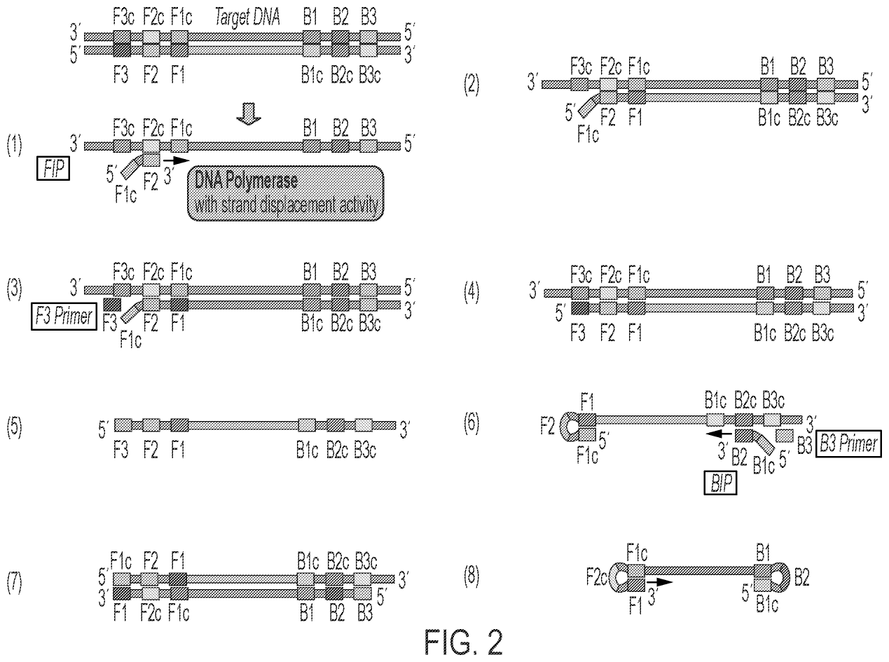

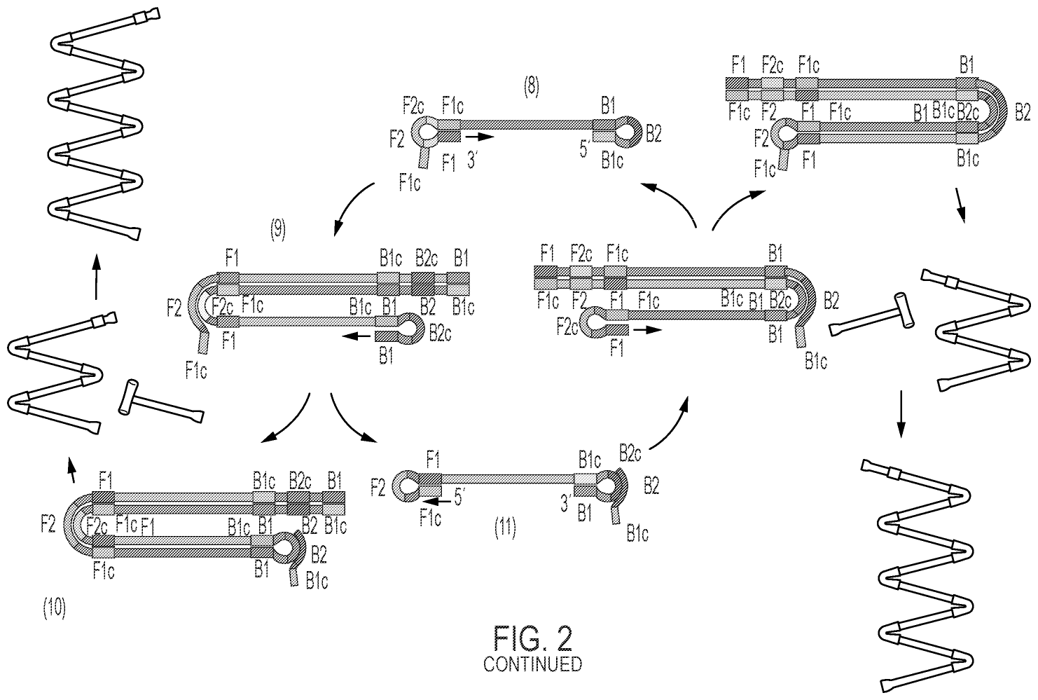

Traditional LAMP technologies copy and/or amplify a templated nucleic acid synthesis target under isothermal conditions using two or more sets of primers (e.g., a pluarality) and a polymerase with high strand displacement activity (e.g., as described in exemplary LAMP reaction below and/or shown in FIG. 2). Further, LAMP templated nucleic acid synthesis reactions can be conducted in a single reaction vessel (“one-pot” embodiment). Typically, four different primers are used, a Forward Primer (F3), a Backward Primer (B3), a Forward Inner Primer (FIP), and/or a Backward Inner Primer (BIP). Both a FIP and/or BIP contact complementary sequences nested within complementary sequences that F3 and/or B3 contact. In some embodiments, a primer includes a single promoter sequence. Optionally, an additional pair of primers (e.g., loop primers) can be used that hybridize to stem-loops (e.g., as discussed below, step 5), except for loops that are hybridized by an inner primer. Use of loop primers can increase LAMP product generated and/or decrease duration of a reaction required to achieve a detection limit. The present disclosure, among other things, provides improved and/or alterative LAMP technologies and/or systems for templated nucleic acid synthesis and/or detecting nucleic acid in a sample (FIG. 1).

### Traditional LAMP Reaction

As those skilled in the art will be aware, a typical LAMP reaction often involves steps such as primer annealing and initiation of templated nucleic acid synthesis. For example, a representative LAMP reaction, depicted in FIG. 2, includes steps of:

STEP 1: Double stranded DNA (e.g., templated nucleic acid synthesis target) is contacted with a LAMP primer (“FIP” in FIG. 2) under conditions (e.g., salt, temperature, etc.) that permit annealing to a complimentary sequence in the DNA. Those skilled in the art will appreciate that available LAMP technologies (e.g., as described in WO2002024902A1) achieve annealing under conditions where complete denaturation is not expected to occur. Without wishing to be bound by any particular theory, it has been proposed that dynamic equilibrium of the double stranded DNA is sufficient to permit such annealing, and will be familiar with conditions (e.g., a temperature within a range of about 60 to about 65° C., and in many embodiments around 65° C.) at which appropriate dynamic equilibrium characteristics occur.

STEP 2: A nucleic acid polymerase enzyme (e.g., with strand displacement activity) extends the primer, synthesizing a new strand that displaces the original strand. Those skilled in the art will appreciate that developed LAMP technologies can achieve templated nucleic acid synthesis even without prior denaturation (e.g., heat denaturation) of the original double stranded DNA.

STEP 3: Double stranded DNA is contacted with an additional LAMP primer (“F3” in FIG. 2) that anneals to a region on a templated nucleic acid synthesis target outside of that which a “FIP” primer annealed. A nucleic acid polymerase enzyme (e.g., with stand displacement activity) extends from a “F3” primer, displacing and releasing a “FIP”-linked complementary strand.

STEP 4: A double strand is formed as a result of DNA synthesized from a “F3” primer along with the remaining, non-displaced original template (e.g., templated nucleic acid synthesis target) DNA strand.

STEP 5: A “FIP”-linked complementary strand, which was released due to DNA synthesized from a “F3” primer, comprises complementary regions at the 5′ end, resulting in formation of a stem-loop structure at said 5′ end.

STEP 6: A single-stranded, stem-loop comprising structure (from Step 5 of FIG. 2) is contacted with an additional LAMP primer (“BIP” in FIG. 2) under conditions (e.g., salt, temperature, etc.) that permit annealing to a complimentary sequence in the DNA. Synthesis of complementary DNA occurs from the 3′ end of a “BIP”, reverting said loop structure into a linear structure. An additional LAMP primer (“B3” in FIG. 2) anneals to a region on a templated nucleic acid synthesis target outside of that which a “BIP” primer annealed. A nucleic acid polymerase (e.g., with strand displacement activity) extends from a “B3” primer, displacing a “BIP”-linked complementary strand.

STEP 7: A dsDNA is produced via STEP 6.

STEP 8: A displaced “BIP”-linked complementary strand forms a structure with stem-loops at each end (e.g., a “dumbbell” structure). The dumbbell structure can serve as a starting structure for cycling templated nucleic acid synthesis (e.g., amplification)

STEPS 8-11 (Cycling templated nucleic acid synthesis step): A dumbbell-like DNA structure is converted into a stem-loop DNA by self-primed DNA synthesis. A “FIP” anneals to the single stranded region in the stem-loop DNA and primes strand displacement DNA synthesis, releasing the strand synthesized as a result of self-priming. The released single strand forms a stem-loop structure at the 3′ end due to complementary regions. Then, starting from the 3′ end, a nucleic acid polymerase (e.g., with stand displacement activity) extends, using self-structure as a template, and releases a “FIP”-linked complementary strand (STEP 9). The released single strand then forms a dumbbell-like structure (STEP 11). Similar to the Steps from 8 to 11, structure in STEP 11 of FIG. 2 leads to self-primed DNA synthesis starting from the 3′ end. A “BIP” anneals and primes strand displacement DNA synthesis, releasing an additional DNA strand. Accordingly, similar structures to Steps 9 and 10 as well as the same structure as Step 8 from FIG. 2 are produced. With the structure produced in Step 10, a “BIP” anneals to a single stranded region, and DNA synthesis continues by displacing double stranded DNA sequence. As a result, various sized structures consisting of alternately inverted repeats of the target sequence on the same strand are formed.

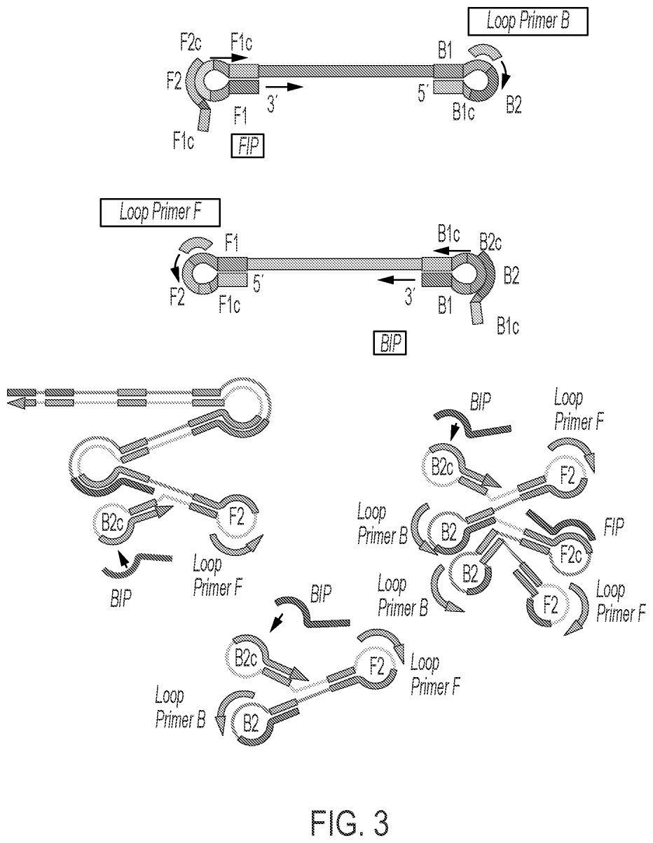

In some embodiments, a LAMP reaction can further utilize loop primers (forward and/or backward) which comprise nucleic acid sequences complementary to the single stranded loop region produced in the described exemplary LAMP reaction. Use of loop primers provides an increased number of starting points for templated nucleic acid synthesis for LAMP, increasing LAMP product generated and/or decreasing the duration of time required for detecting a nucleic acid. An exemplary templated nucleic acid synthesis further utilizing a forward loop primer (LoopF) and/or a backward loop primer (LoopB) is shown in FIG. 3.

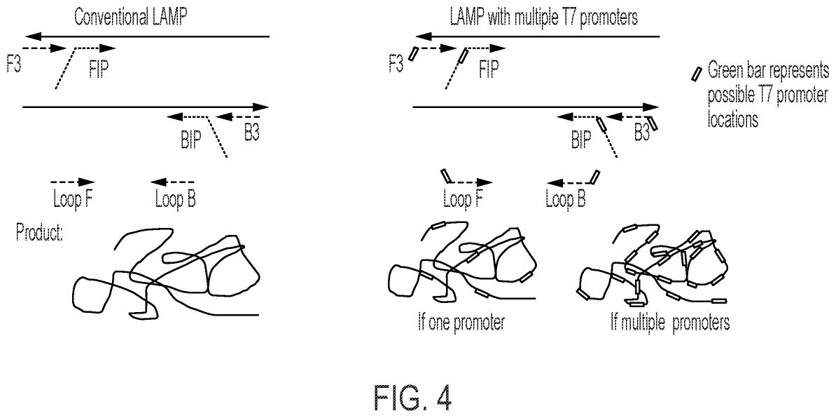

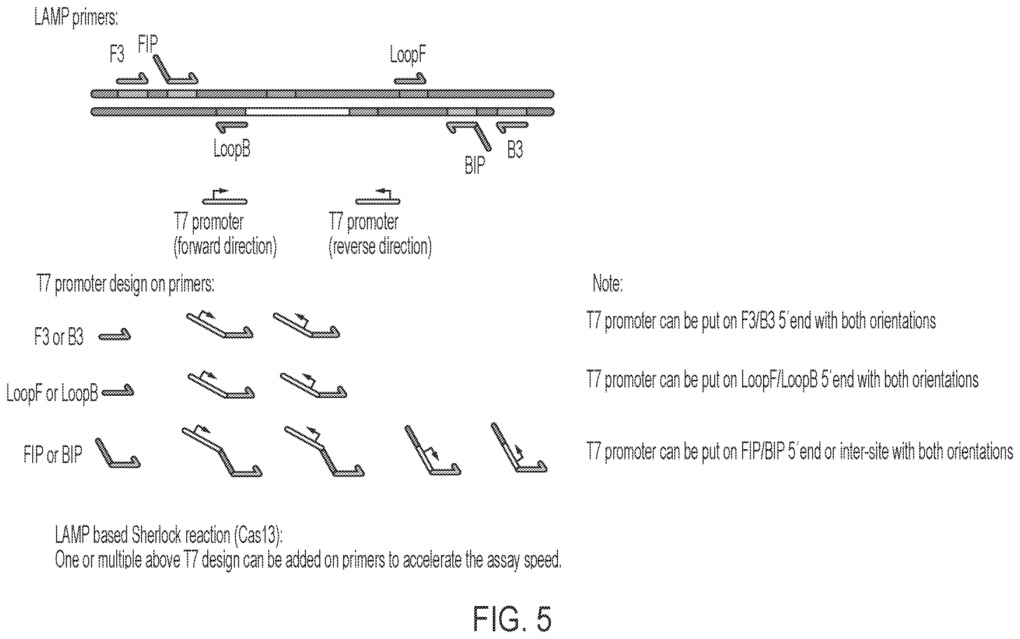

Thus, typically at least one primer is utilized in a LAMP reaction. In fact, typically a plurality of primers are utilized, which may, for example include one or more of a Forward Primer (F3), a Backward Primer (B3), a Forward Inner Primer (FIP), a Backward Inner Primer (BIP), a Forward Loop primer (LoopF), and/or a Backward Loop primer (LoopB) (FIG. 4-5).

Among other things, the present disclosure documents successful templated nucleic acid synthesis and/or detection can be achieved in some embodiments even without any promoter in any primer. In some particular embodiments, promoter(s) are not included in any of a F3, a B3, a FIP, a BIP, a LoopF, and/or a LoopB primer.

Alternatively or additionally, in some embodiments, the present disclosure documents that use of multiple promoters (e.g., multiple promoters within a single LAMP primer and/or at least one promoter on each of at least two LAMP primers) can achieve improved sensitivity and/or reaction speed as well as increase total LAMP product generated (FIG. 4). In some particular, multiple-promoter embodiments, promoter(s) are included in two or more of a Forward Primer (F3), a Backward Primer (B3), a Forward Inner Primer (FIP), a Backward Inner Primer (BIP), a Forward Loop Primer (LoopF), and/or a Backward Loop Primer (LoopB) (FIG. 5). In some embodiments, multiple promoters are included within a single LAMP primer and/or at least one promoter on each of at least two LAMP primers. In some embodiments, a promoter is oriented in the forward direction. In some embodiments, a promoter is oriented in the reverse direction. In some embodiments, an included promoter (or promoters) is a T7 promoter (FIG. 5).

### Exemplary Technologies for Converting Nucleic Acid Subtypes

In some embodiments, a templated nucleic acid synthesis technique requires a particular type of nucleic acid (e.g., ssDNA, dsDNA, ssRNA) starting material (e.g., substrate). In some embodiments, a templated nucleic acid synthesis target may need to be converted to a different type of nucleic acid prior to templated nucleic acid synthesis (e.g., conversion of ssRNA to dsDNA by reverse transcription). For example, LAMP substrates are dsDNA.

In some embodiments, wherein a templated nucleic acid synthesis target is ssDNA, a reaction to convert ssDNA to dsDNA is conducted. In some embodiments, ssDNA is converted to dsDNA by any method known to one of ordinary skill in the art, for example, polymerase chain reaction (PCR) or Klenow reaction.

In some embodiments, wherein a templated nucleic acid synthesis target is a RNA, a RNA is converted to dsDNA by any method known to one of ordinary skill in the art, for example, by a reverse transcription reaction, prior to templated nucleic acid synthesis. In some embodiments, wherein a templated nucleic acid synthesis target is RNA, RNA is converted to dsDNA prior to templated nucleic acid synthesis by LAMP reaction.

### Transcription of LAMP Product

In some embodiments, provided technologies comprise transcribing a copied and/or amplified templated nucleic acid synthesis target using any primer inserted promoter (FIG. 1, 4, 5). In some embodiments, a primer inserted promoter is a T7 promoter. In some embodiments, a copied and/or amplified templated nucleic acid synthesis target may be amplified (or further amplified) by generating RNA (e.g., by transcription of LAMP product).

In some particular embodiments, a promoter is not included in any of a F3, a B3, a FIP, a BIP, a LoopF, and/or a LoopB primer, yet LAMP product can still be transcribed. Without wishing to be bound by any particular theory, it is hypothesized due to promiscuity of RNA polymerases, transcription can occur without a promoter, though at lower efficiency than transcription which occurs from a promoter sequence.

In some embodiments, templated nucleic acid synthesis (e.g., by LAMP) and transcription of a nucleic acid, can occur in a one-pot method.

### Detection of Transcript

One of skill in the art is aware of various technologies useful in detecting nucleic acids. In some embodiments, the present disclosure provides technologies for detecting transcript. In some embodiments, detection technologies comprise, for example, absorbance, CRISPR/Cas detection (e.g., CRISPR-SHERLOCK), FRET, gel electrophoresis, lateral flow, mass spectrometry, PCR, real-time PCR, and/or spectrometry. In some embodiments, detection technologies comprise, for example, chemiluminescence, electrochemical technologies, fluorescence, intercalating dye detection, migration, and/or radiation.

Certain CRISPR/Cas enzymes have been identified that have an ability to non-specifically cleave collateral nucleic acid(s) when activated by binding to a target site recognized by the guide RNA with which they are complexed (e.g., Cas target nucleic acid). Representative examples of Cas12, Cas13, and Cas14 enzymes have been shown to have such collateral cleavage activity. See, for example, Swarts and Jinek Mol Cell. 2019 Feb. 7; 73(3):589-600.e4; Harrington L. B. et al. Science. 2018; 362:839-842; Li S. Y. et al. Cell Res. 2018; 28:491-493; Chen J. S. et al., Science. 2018; 360:436-439; Abudayyeh O. O. et al., Science. 2016; 353aaf5573; East-Seletsky A et al., Nature. 2016; 538:270-273; Gootenberg J S et al.; Science 2017; 356:438-442; Myhrvold C, et al., Science 2018; 360:444-448; Gootenberg J S et al., Science 2018; 360:439-444. Some CRISPR/Cas enzyme collateral cleavage activity digests or cleaves single strand nucleic acids. Some CRISPR/Cas enzyme collateral cleavage activity digests or cleaves double stranded nucleic acids. Some CRISPR/Cas enzyme collateral cleavage activity digests or cleaves RNA. Some CRISPR/Cas enzyme collateral cleavage activity digests or cleaves DNA. Some CRISPR/Cas enzyme collateral cleavage activity digests or cleaves both RNA and DNA. Collateral activity has been harnessed to develop CRISPR/Cas detection (e.g., diagnostic) technologies that achieve detection of nucleic acids containing the relevant target site (e.g., Cas target nucleic acid), or its complement, in biological and/or environmental sample(s). See, for example Gootenberg J S et al.; Science 2017; 356:438-442; WO2019/011022; U.S. Pat. No. 10,494,664B2; U.S. Pat. No. 10,337,051B2; U.S. Pat. No. 10,266,887; sherlock.bio/better-faster-affordable-diagnostic-testing.

CRISPR-SHERLOCK is a detection technology comprising steps of: contacting a CRISPR-Cas complex comprising a Cas protein with collateral cleavage activity, a guide RNA selected or engineered to be complementary to a target sequence (e.g., a Cas target nucleic acid sequence), and a sample potentially comprising a Cas target nucleic acid (see, e.g., WO 2018/107129, WO 2019/011022, incorporated herein by reference). In some embodiments, CRISPR/Cas-based detection may be a CRISPR-Cas13-based detection system. In some embodiments, a CRISPR/Cas-based detection system is a CRISPR/Cas12-based detection system. In some embodiments, a CRISPR/Cas13- or CRISPR/Cas12-based detection system is a CRISPR-SHERLOCK detection system.

### Compositions

The present disclosure provides LAMP components and/or combinations thereof, and/or systems, e.g., that may include a target (e.g., templated nucleic acid synthesis target, copied and/or amplified templated nucleic acid synthesis target, Cas taget nucleic acid), and/or other processing components (e.g., SHERLOCK and/or any other suitable detection method). In some embodiments the present disclosure provides compositions and/or components useful for templated nucleic acid synthesis. In some embodiments, the present disclosure provides compositions and/or components for LAMP. In some, embodiments, the present disclosure provides compositions and/or components for template nucleic acid synthesis and/or detection of Cas target nucleic acids and/or compositions thereof.

### Template Nucleic Acid and Sources of Template Nucleic Acids (e.g., Samples)

A template (e.g., for templated nucleic acid synthesis) DNA or RNA may be a DNA or RNA or a part of a DNA or RNA to which a contacting nucleic acid or nucleic acids (e.g., primer(s)) have complementarity. In some embodiments, a template nucleic acid may be double-stranded. In some embodiments, a template nucleic acid may be single-stranded). In some embodiments, a template nucleic acid may be genomic DNA, mitochondrial DNA, viral DNA, plasmid DNA, synthetic dsDNA, or RNA. In some embodiments, a single-stranded nucleic acid comprises single-stranded viral DNA, viral RNA, messenger RNA, ribosomal RNA, transfer RNA, microRNA, short interfering RNA, small nuclear RNA, synthetic RNA, and/or synthetic ssDNA.

In some embodiments, a templated nucleic acid synthesis target is in and/or isolated from a sample. In some embodiments, a sample may include, but is not limited to, a biological sample or an environmental sample, such as a food sample (fresh fruits or vegetables, meats), a beverage sample, a fabric surface, a freshwater sample, a paper surface, a plastic surface, a metal surface, a soil sample, a saline water sample, a wastewater sample, a wood surface, exposure to atmospheric air or other gas sample, or a combination thereof. Further examples include, household/commercial/industrial surfaces which may be made of any materials including, but not limited to, metal, wood, plastic, rubber, or the like, and may be swabbed and/or tested for contaminants. Soil samples may be tested for the presence of pathogenic bacteria or parasites, or other microbes, both for environmental purposes and/or for human, animal, or plant disease testing.

In some embodiments, a biological sample may be obtained from a source including, but not limited to, ascites, blood, bone, cerebrospinal fluid, plasma, pleural effusion, fecal matter, hair follicle, pus, lymph, mucous, muscle, nasal fluid, saliva, semen, sera, seroma, skin, sputum, stool, synovial fluid, a tissue sample, teeth, urine, or a swab of a skin or a mucosal membrane surface. In some embodiments, an environmental sample or biological sample may be crude samples and/or the one or more target molecules may not be purified or amplified from the sample prior to the application of the technologies. Identification of microbes may be useful and/or needed for any number of applications, and thus any type of sample from any source deemed appropriate by one of skill in the art may be used in accordance with the invention.

### Polymerases

Disclosed technologies utilize a polymerase, for example, for templated nucleic acid synthesis, conversion of one nucleic acid type to another, and/or transcription. In some embodiments, a DNA polymerase is utilized for templated nucleic acid synthesis (e.g., a DNA polymerase with high strand displacement activity). In some embodiments, wherein the templated nucleic acid synthesis target is a RNA, a reverse transcriptase can first be used to copy a RNA target into a cDNA molecule suitable for templated nucleic acid synthesis (e.g., LAMP). In some embodiments, disclosed technologies utilize an RNA polymerase to transcribe templated nucleic acid synthesis product (e.g., LAMP product). In some embodiments, provided technologies utilize both a DNA and a RNA polymerase. A polymerase useful in accordance with the disclosed technologies may be any specific or general polymerase known in the art and/or useful.

In some embodiments, a LAMP reaction utilizes a DNA polymerase enzyme, preferably a DNA polymerase with high strand displacement activity. In some embodiments, templated nucleic acid synthesis (e.g., by LAMP) is followed by a transcription reaction, wherein a copied and/or amplified target sequence is transcribed by an RNA polymerase. In some embodiments, template nucleic acid synthesis and transcription occur in a single reaction vessel (“one-pot”).

### DNA Polymerases

In some embodiments, templated nucleic acid synthesis utilizes a DNA polymerase. In some embodiments, a DNA polymerase utilized has high strand displacement activity (e.g., the ability to displace downstream DNA encountered during synthesis). In some embodiments, a polymerase can be selected from, for example, Bst 2.0 DNA polymerase, Bst 2.0 WarmStart DNA polymerase, Bst 3.0 DNA polymerase, full length Bst DNA polymerase, large fragment Bst DNA polymerase, large fragment Bsu DNA polymerase, Klenow fragment of E. coli DNA polymerase I, KlenTaq, Gst polymerase, phi29 DNA polymerase, Pol III DNA polymerase, T5 DNA polymerase, T7 DNA polymerase, Taq polymerase, and Sequenase DNA polymerase.

Template nucleic acid synthesis can be isothermal and selected for temperature. Isothermal reactions typically refer to reactions performed without drastic temperature cycling, e.g., without temperature fluctuations of more than about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C., or temperature fluctuations less than about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. In some embodiments, templated nucleic acid synthesis can be performed at about 60-65° C.

In some embodiments, a useful polymerase performs (e.g., synthesizes nucleic acids) at temperatures above about 50° C.; in some embodiments, above a temperature selected from the group consisting of about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., or combinations thereof. In many embodiments, useful polymerase performs (e.g., synthesizes nucleic acids) at temperatures above about 60° C.

In some embodiments, a templated nucleic acid synthesis is performed within a temperature range at which a useful polymerase (e.g., synthesizes nucleic acids) performs. In some embodiments, a useful polymerase performs (e.g., synthesizes nucleic acids) within a temperature range at which templated nucleic acid synthesis is performed. Those skilled in the art are well familiar with various such reactions and the temperature ranges at which they are performed. In some embodiments, such a temperature range may be above a temperature selected from the group consisting of about 60° C., about 61° C., about 62° C., about 63° C., about 64° C., 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., or combinations thereof. In some embodiments, a temperature range may be about 60° C. to about 90° C. In some embodiments, a temperature range may be about 60° C. to about 80° C. In some embodiments, a temperature range may be about 60° C. to about 75° C. In some embodiments, a temperature range may be about 65° C. to about 90° C. In some embodiments, a temperature range may be about 60° C. to about 80° C. In some embodiments, a temperature range may be about 60° C. to about 65° C.

### RNA Polymerases

In some embodiments a nucleic acid is transcribed using an RNA polymerase. In some embodiments, an RNA polymerase utilized is a DNA-dependent RNA polymerase. In some embodiments, an RNA polymerase is, for example, T7 or SP6. In some embodiments, an RNA polymerase utilized can synthesize RNA in the absence of a promoter sequence. One of ordinary skill in the art would readily appreciate that any suitable promoter and/or a polymerase capable of recognizing said promoter could be utilized.

### T7

T7 is a DNA-dependent RNA polymerase (T7RNAP) first isolated from bacteriophage T7-infected Escherichia coli cells. T7RNAP is a single-subunit enzyme with high specificity toward the T7 promoter and does not need any additional protein factors to perform the complete transcriptional cycle. T7RNAP has proven to be useful in various in vitro contexts and/or reactions, including, but not limited to, large-scale production processes, expression systems, inducible expression systems, RNA editing, and/or RNA interference. T7RNAP, and variants thereof, are commercially available from a variety of vendors (for example, New England Biolabs, ThermoFisher, Promega, Bio-rad, Sigma Aldrich, Takara Bio).

In some embodiments, a T7RNAP useful in accordance with the present disclosure may be or comprise an N-terminal domain and a polymerase domain, or a fragment thereof. In some embodiments, an amino acid sequence of a T7RNAP comprises a mutation or variant. In some embodiments, an amino acid sequence of a T7RNAP comprises a mutation or variant with altered specificity and/or activity relative to an appropriate reference (e.g., wild-type T7RNAP). In some embodiments, a nucleic acid sequence T7RNAP may comprise a codon optimized sequence. In some embodiments, a T7RNAP polymerase may be encoded by a homolog or ortholog of a T7RNAP sequence. In some embodiments, a homolog or ortholog of a T7RNAP as referred to herein has a sequence homology or identity of at least 80%, at least 85%, at least 90%, or at least 95% with a T7RNAP sequence (SEQ ID NOs: 69-70).

T7RNAPs have been shown to initiate transcription from promoters characterized by a highly conserved nucleic acid sequence (e.g., SEQ ID NO. 72). T7RNAPs transcribe using the opposite strand of the T7 promoter as a template from 5′ to 3′. Typically, T7 promoters are characterized by DNA sequences approximately 18-23 base pairs long up to a transcription start site at +1 position and is recognized by T7 RNAPs.

In some embodiments, a promoter for use as described herein is 18, 19, 20, 21, 22, or 23 nucleotides in length. In some embodiments, a T7 promoter nucleotide sequence comprises a variant or mutation which alters binding or recognition by a T7RNAP. In some embodiments a T7 promoter may have a codon optimized nucleic acid sequence.

Exemplary RNAPs

| SEQ | | |
|---|---|---|
| ID NO. | Polymerase | Sequence |
| 69 | T7 RNAP | TCGCGCTGCACTGGCGTAATGCTGACCGGATGGCTATCGCT |
| | | AATGGTCTTACGCTCAACATTGATAAGCAACTTGACGCAAT |
| | | GTTAATGGGCTGATAGTCTTATCTTACAGGTCATCTGCGGGT |
| | | GGCCTGAATAGGTACGATTTACTAACTGGAAGAGGCACTAA |
| | | ATGAACACGATTAACATCGCTAAGAACGACTTCTCTGACAT |
| | | CGAACTGGCTGCTATCCCGTTCAACACTCTGGCTGACCATTA |
| | | CGGTGAGCGTTTAGCTCGCGAACAGTTGGCCCTTGAGCATG |
| | | AGTCTTACGAGATGGGTGAAGCACGCTTCCGCAAGATGTTT |
| | | GAGCGTCAACTTAAAGCTGGTGAGGTTGCGGATAACGCTGC |
| | | CGCCAAGCCTCTCATCACTACCCTACTCCCTAAGATGATTGC |
| | | ACGCATCAACGACTGGTTTGAGGAAGTGAAAGCTAAGCGC |
| | | GGCAAGCGCCCGACAGCCTTCCAGTTCCTGCAAGAAATCA |
| | | AGCCGGAAGCCGTAGCGTACATCACCATTAAGACCACTCTG |
| | | GCTTGCCTAACCAGTGCTGACAATACAACCGTTCAGGCTGT |
| | | AGCAAGCGCAATCGGTCGGGCCATTGAGGACGAGGCTCGC |
| | | TTCGGTCGTATCCGTGACCTTGAAGCTAAGCACTTCAAGAA |
| | | AAACGTTGAGGAACAACTCAACAAGCGCGTAGGGCACGTC |
| | | TACAAGAAAGCATTTATGCAAGTTGTCGAGGCTGACATGCT |
| | | CTCTAAGGGTCTACTCGGTGGCGAGGCGTGGTCTTCGTGGC |
| | | ATAAGGAAGACTCTATTCATGTAGGAGTACGCTGCATCGAG |
| | | ATGCTCATTGAGTCAACCGGAATGGTTAGCTTACACCGCCA |
| | | AAATGCTGGCGTAGTAGGTCAAGACTCTGAGACTATCGAAC |
| | | TCGCACCTGAATACGCTGAGGCTATCGCAACCCGTGCAGGT |
| | | GCGCTGGCTGGCATCTCTCCGATGTTCCAACCTTGCGTAGTT |
| | | CCTCCTAAGCCGTGGACTGGCATTACTGGTGGTGGCTATTG |
| | | GGCTAACGGTCGTCGTCCTCTGGCGCTGGTGCGTACTCACA |
| | | GTAAGAAAGCACTGATGCGCTACGAAGACGTTTACATGCCT |
| | | GAGGTGTACAAAGCGATTAACATTGCGCAAAACACCGCATG |
| | | GAAAATCAACAAGAAAGTCCTAGCGGTCGCCAACGTAATC |
| | | ACCAAGTGGAAGCATTGTCCGGTCGAGGACATCCCTGCGAT |
| | | TGAGCGTGAAGAACTCCCGATGAAACCGGAAGACATCGAC |
| | | ATGAATCCTGAGGCTCTCACCGCGTGGAAACGTGCTGCCGC |
| | | TGCTGTGTACCGCAAGGACAGGGCTCGCAAGTCTCGCCGTA |
| | | TCAGCCTTGAGTTCATGCTTGAGCAAGCCAATAAGTTTGCT |
| | | AACCATAAGGCCATCTGGTTCCCTTACAACATGGACTGGCG |
| | | CGGTCGTGTTTACGCCGTGTCAATGTTCAACCCGCAAGGTA |
| | | ACGATATGACCAAAGGACTGCTTACGCTGGCGAAAGGTAAA |
| | | CCAATCGGTAAGGAAGGTTACTACTGGCTGAAAATCCACGG |
| | | TGCAAACTGTGCGGGTGTCGATAAGGTTCCGTTCCCTGAGC |
| | | GCATCAAGTTCATTGAGGAAAACCACGAGAACATCATGGCT |
| | | TGCGCTAAGTCTCCACTGGAGAACACTTGGTGGGCTGAGC |
| | | AAGATTCTCCGTTCTGCTTCCTTGCGTTCTGCTTTGAGTACG |
| | | CTGGGGTACAGCACCACGGCCTGAGCTATAACTGCTCCCTT |
| | | CCGCTGGCGTTTGACGGGTCTTGCTCTGGCATCCAGCACTT |
| | | CTCCGCGATGCTCCGAGATGAGGTAGGTGGTCGCGCGGTTA |
| | | ACTTGCTTCCTAGTGAGACCGTTCAGGACATCTACGGGATT |
| | | GTTGCTAAGAAAGTCAACGAGATTCTACAAGCAGACGCAAT |
| | | CAATGGGACCGATAACGAAGTAGTTACCGTGACCGATGAGA |
| | | ACACTGGTGAAATCTCTGAGAAAGTCAAGCTGGGCACTAA |
| | | GGCACTGGCTGGTCAATGGCTGGCTCACGGTGTTACTCGCA |
| | | GTGTGACTAAGCGTTCAGTCATGACGCTGGCTTACGGGTCC |
| | | AAAGAGTTCGGCTTCCGTCAACAAGTGCTGGAAGATACCAT |
| | | TCAGCCAGCTATTGATTCCGGCAAGGGTCCGATGTTCACTC |
| | | AGCCGAATCAGGCTGCTGGATACATGGCTAAGCTGATTTGG |
| | | GAATCTGTGAGCGTGACGGTGGTAGCTGCGGTTGAAGCAAT |
| | | GAACTGGCTTAAGTCTGCTGCTAAGCTGCTGGCTGCTGAGG |
| | | TCAAAGATAAGAAGACTGGAGAGATTCTTCGCAAGCGTTGC |
| | | GCTGTGCATTGGGTAACTCCTGATGGTTTCCCTGTGTGGCA |
| | | GGAATACAAGAAGCCTATTCAGACGCGCTTGAACCTGATGT |
| | | TCCTCGGTCAGTTCCGCTTACAGCCTACCATTAACACCAAC |
| | | AAAGATAGCGAGATTGATGCACACAAACAGGAGTCTGGTAT |
| | | CGCTCCTAACTTTGTACACAGCCAAGACGGTAGCCACCTTC |
| | | GTAAGACTGTAGTGTGGGCACACGAGAAGTACGGAATCGA |
| | | ATCTTTTGCACTGATTCACGACTCCTTCGGTACCATTCCGGC |
| | | TGACGCTGCGAACCTGTTCAAAGCAGTGCGCGAAACTATG |
| | | GTTGACACATATGAGTCTTGTGATGTACTGGCTGATTTCTAC |
| | | GACCAGTTCGCTGACCAGTTGCACGAGTCTCAATTGGACAA |
| | | AATGCCAGCACTTCCGGCTAAAGGTAACTTGAACCTCCGTG |
| | | ACATCTTAGAGTCGGACTTCGCGTTCGCGTAACGCCAAATC |
| | | AATACGACTCACTATAGAGGGACAAACTCAAGGTCATTCGC |
| | | AAGAGTGGCC |
| 70 | T7RNAP | ATGAACACGATTAACATCGCTAAGAACGACTTCTCTGACAT |
| | | CGAACTGGCTGCTATCCCGTTCAACACTCTGGCTGACCATTA |
| | | CGGTGAGCGTTTAGCTCGCGAACAGTTGGCCCTTGAGCATG |
| | | AGTCTTACGAGATGGGTGAAGCACGCTTCCGCAAGATGTTT |
| | | GAGCGTCAACTTAAAGCTGGTGAGGTTGCGGATAACGCTGC |
| | | CGCCAAGCCTCTCATCACTACCCTACTCCCTAAGATGATTGC |
| | | ACGCATCAACGACTGGTTTGAGGAAGTGAAAGCTAAGCGC |
| | | GGCAAGCGCCCGACAGCCTTCCAGTTCCTGCAAGAAATCA |
| | | AGCCGGAAGCCGTAGCGTACATCACCATTAAGACCACTCTG |
| | | GCTTGCCTAACCAGTGCTGACAATACAACCGTTCAGGCTGT |
| | | AGCAAGCGCAATCGGTCGGGCCATTGAGGACGAGGCTCGC |
| | | TTCGGTCGTATCCGTGACCTTGAAGCTAAGCACTTCAAGAA |
| | | AAACGTTGAGGAACAACTCAACAAGCGCGTAGGGCACGTC |
| | | TACAAGAAAGCATTTATGCAAGTTGTCGAGGCTGACATGCT |
| | | CTCTAAGGGTCTACTCGGTGGCGAGGCGTGGTCTTCGTGGC |
| | | ATAAGGAAGACTCTATTCATGTAGGAGTACGCTGCATCGAG |
| | | ATGCTCATTGAGTCAACCGGAATGGTTAGCTTACACCGCCA |
| | | AAATGCTGGCGTAGTAGGTCAAGACTCTGAGACTATCGAAC |
| | | TCGCACCTGAATACGCTGAGGCTATCGCAACCCGTGCAGGT |
| | | GCGCTGGCTGGCATCTCTCCGATGTTCCAACCTTGCGTAGTT |
| | | CCTCCTAAGCCGTGGACTGGCATTACTGGTGGTGGCTATTG |
| | | GGCTAACGGTCGTCGTCCTCTGGCGCTGGTGCGTACTCACA |
| | | GTAAGAAAGCACTGATGCGCTACGAAGACGTTTACATGCCT |
| | | GAGGTGTACAAAGCGATTAACATTGCGCAAAACACCGCATG |
| | | GAAAATCAACAAGAAAGTCCTAGCGGTCGCCAACGTAATC |
| | | ACCAAGTGGAAGCATTGTCCGGTCGAGGACATCCCTGCGAT |
| | | TGAGCGTGAAGAACTCCCGATGAAACCGGAAGACATCGAC |
| | | ATGAATCCTGAGGCTCTCACCGCGTGGAAACGTGCTGCCGC |
| | | TGCTGTGTACCGCAAGGACAAGGCTCGCAAGTCTCGCCGTA |
| | | TCAGCCTTGAGTTCATGCTTGAGCAAGCCAATAAGTTTGCT |
| | | AACCATAAGGCCATCTGGTTCCCTTACAACATGGACTGGCG |
| | | CGGTCGTGTTTACGCTGTGTCAATGTTCAACCCGCAAGGTA |
| | | ACGATATGACCAAAGGACTGCTTACGCTGGCGAAAGGTAAA |
| | | CCAATCGGTAAGGAAGGTTACTACTGGCTGAAAATCCACGG |
| | | TGCAAACTGTGCGGGTGTCGATAAGGTTCCGTTCCCTGAGC |
| | | GCATCAAGTTCATTGAGGAAAACCACGAGAACATCATGGCT |
| | | TGCGCTAAGTCTCCACTGGAGAACACTTGGTGGGCTGAGC |
| | | AAGATTCTCCGTTCTGCTTCCTTGCGTTCTGCTTTGAGTACG |
| | | CTGGGGTACAGCACCACGGCCTGAGCTATAACTGCTCCCTT |
| | | CCGCTGGCGTTTGACGGGTCTTGCTCTGGCATCCAGCACTT |
| | | CTCCGCGATGCTCCGAGATGAGGTAGGTGGTCGCGCGGTTA |
| | | ACTTGCTTCCTAGTGAAACCGTTCAGGACATCTACGGGATT |
| | | GTTGCTAAGAAAGTCAACGAGATTCTACAAGCAGACGCAAT |
| | | CAATGGGACCGATAACGAAGTAGTTACCGTGACCGATGAGA |
| | | ACACTGGTGAAATCTCTGAGAAAGTCAAGCTGGGCACTAA |
| | | GGCACTGGCTGGTCAATGGCTGGCTTACGGTGTTACTCGCA |
| | | GTGTGACTAAGCGTTCAGTCATGACGCTGGCTTACGGGTCC |
| | | AAAGAGTTCGGCTTCCGTCAACAAGTGCTGGAAGATACCAT |
| | | TCAGCCAGCTATTGATTCCGGCAAGGGTCTGATGTTCACTC |
| | | AGCCGAATCAGGCTGCTGGATACATGGCTAAGCTGATTTGG |
| | | GAATCTGTGAGCGTGACGGTGGTAGCTGCGGTTGAAGCAAT |
| | | GAACTGGCTTAAGTCTGCTGCTAAGCTGCTGGCTGCTGAGG |
| | | TCAAAGATAAGAAGACTGGAGAGATTCTTCGCAAGCGTTGC |
| | | GCTGTGCATTGGGTAACTCCTGATGGTTTCCCTGTGTGGCA |
| | | GGAATACAAGAAGCCTATTCAGACGCGCTTGAACCTGATGT |
| | | TCCTCGGTCAGTTCCGCTTACAGCCTACCATTAACACCAAC |
| | | AAAGATAGCGAGATTGATGCACACAAACAGGAGTCTGGTAT |
| | | CGCTCCTAACTTTGTACACAGCCAAGACGGTAGCCACCTTC |
| | | GTAAGACTGTAGTGTGGGCACACGAGAAGTACGGAATCGA |
| | | ATCTTTTGCACTGATTCACGACTCCTTCGGTACCATTCCGGC |
| | | TGACGCTGCGAACCTGTTCAAAGCAGTGCGCGAAACTATG |
| | | GTTGACACATATGAGTCTTGTGATGTACTGGCTGATTTCTAC |
| | | GACCAGTTCGCTGACCAGTTGCACGAGTCTCAATTGGACAA |
| | | AATGCCAGCACTTCCGGCTAAAGGTAACTTGAACCTCCGTG |
| | | ACATCTTAGAGTCGGACTTCGCGTTCGCGTAATAA |
| 71 | SP6 | GCGCTCAATTAAGTTTTCTAGTACCGCATGAGGATACAAGAT |
| | | GCAAGATTTACACGCTATCCAGCTTCAATTAGAAGAAGAGA |
| | | TGTTTAATGGTGGCATTCGTCGCTTCGAAGCAGATCAACAA |
| | | CGCCAGATTGCAGCAGGTAGCGAGAGCGACACAGCATGGA |
| | | ACCGCCGCCTGTTGTCAGAACTTATTGCACCTATGGCTGAA |
| | | GGCATTCAGGCTTATAAAGAAGAGTACGAAGGTAAGAAAG |
| | | GTCGTGCACCTCGCGCATTGGCTTTCTTACAATGTGTAGAAA |
| | | ATGAAGTTGCAGCATACATCACTATGAAAGTTGTTATGGATA |
| | | TGCTGAATACGGATGCTACCCTTCAGGCTATTGCAATGAGTG |
| | | TAGCAGAACGCATTGAAGACCAAGTGCGCTTTTCTAAGCTA |
| | | GAAGGTCACGCCGCTAAATACTTTGAGAAGGTTAAGAAGTC |
| | | ACTCAAGGCTAGCCGTACTAAGTCATATCGTCACGCTCATAA |
| | | CGTAGCTGTAGTTGCTGAAAAATCAGTTGCAGAAAAGGAC |
| | | GCGGACTTTGACCGTTGGGAGGCGTGGCCAAAAGAAACTC |
| | | AATTGCAGATTGGTACTACCTTGCTTGAAATCTTAGAAGGTA |
| | | GCGTTTTCTATAATGGTGAACCTGTATTTATGCGTGCTATGCG |
| | | CACTTATGGCGGAAAGACTATTTACTACTTACAAACTTCTGA |
| | | AAGTGTAGGCCAGTGGATTAGCGCATTCAAAGAGCACGTAG |
| | | CGCAATTAAGCCCAGCTTATGCCCCTTGCGTAATCCCTCCTC |
| | | GTCCTTGGAGAACTCCATTTAATGGAGGGTTCCATACTGAG |
| | | AAGGTAGCTAGCCGTATCCGTCTTGTAAAAGGTAACCGTGA |
| | | GCATGTACGCAAGTTGACTCAAAAGCAAATGCCAAAGGTTT |
| | | ATAAGGCTATCAACGCATTACAAAATACACAATGGCAAATC |
| | | AACAAGGATGTATTAGCAGTTATTGAAGAAGTAATCCGCTTA |
| | | GACCTTGGTTATGGTGTACCTTCCTTCAAGCCACTGATTGAC |
| | | AAGGAGAACAAGCCAGCTAACCCGGTACCTGTTGAATTCCA |
| | | ACACCTGCGCGGTCGTGAACTGAAAGAGATGCTATCACCTG |
| | | AGCAGTGGCAACAATTCATTAACTGGAAAGGCGAATGCGC |
| | | GCGCCTATATACCGCAGAAACTAAGCGCGGTTCAAAGTCCG |
| | | CCGCCGTTGTTCGCATGGTAGGACAGGCCCGTAAATATAGC |
| | | GCCTTTGAATCCATTTACTTCGTGTACGCAATGGATAGCCGC |
| | | AGCCGTGTCTATGTGCAATCTAGCACGCTCTCTCCGCAGTCT |
| | | AACGACTTAGGTAAGGCATTACTCCGCTTTACCGAGGGACG |
| | | CCCTGTGAATGGCGTAGAAGCGCTTAAATGGTTCTGCATCA |
| | | ATGGTGCTAACCTTTGGGGATGGGACAAGAAAACTTTTGAT |
| | | GTGCGCGTGTCTAACGTATTAGATGAGGAATTCCAAGATATG |
| | | TGTCGAGACATCGCCGCAGACCCTCTCACATTCACCCAATG |
| | | GGCTAAAGCTGATGCACCTTATGAATTCCTCGCTTGGTGCTT |
| | | TGAGTATGCTCAATACCTTGATTTGGTGGATGAAGGAAGGG |
| | | CCGACGAATTCCGCACTCACCTACCAGTACATCAGGACGGG |
| | | TCTTGTTCAGGCATTCAGCACTATAGTGCTATGCTTCGCGAC |
| | | GAAGTAGGGGCCAAAGCTGTTAACCTGAAACCCTCCGATGC |
| | | ACCGCAGGATATCTATGGGGCGGTGGCGCAAGTGGTTATCA |
| | | AGAAGAATGCGCTATATATGGATGCGGACGATGCAACCACG |
| | | TTTACTTCTGGTAGCGTCACGCTGTCCGGTACAGAACTGCG |
| | | AGCAATGGCTAGCGCATGGGATAGTATTGGTATTACCCGTAG |
| | | CTTAACCAAAAAGCCCGTGATGACCTTGCCATATGGTTCTAC |
| | | TCGCTTAACTTGCCGTGAATCTGTGATTGATTACATCGTAGA |
| | | CTTAGAGGAAAAAGAGGCGCAGAAGGCAGTAGCAGAAGG |
| | | GCGGACGGCAAACAAGGTACATCCTTTTGAAGACGATCGTC |
| | | AAGATTACTTGACTCCGGGCGCAGCTTACAACTACATGACG |
| | | GCACTAATCTGGCCTTCTATTTCTGAAGTAGTTAAGGCACCG |
| | | ATAGTAGCTATGAAGATGATACGCCAGCTTGCACGCTTTGCA |
| | | GCGAAACGTAATGAAGGCCTGATGTACACCCTGCCTACTGG |
| | | CTTCATCTTAGAACAGAAGATCATGGCAACCGAGATGCTAC |
| | | GCGTGCGTACCTGTCTGATGGGTGATATCAAGATGTCCCTTC |
| | | AGGTTGAAACGGATATCGTAGATGAAGCCGCTATGATGGGA |
| | | GCAGCAGCACCTAATTTCGTACACGGTCATGACGCAAGTCA |
| | | CCTTATCCTTACCGTATGTGAATTGGTAGACAAGGGCGTAAC |
| | | TAGTATCGCTGTAATCCACGACTCTTTTGGTACTCATGCAGA |
| | | CAACACCCTCACTCTTAGAGTGGCACTTAAAGGGCAGATGG |
| | | TTGCAATGTATATTGATGGTAATGCGCTTCAGAAACTACTGG |
| | | AGGAGCATGAAGTGCGCTGGATGGTTGATACAGGTATCGAA |
| | | GTACCTGAGCAAGGGGAGTTCGACCTTAACGAAATCATGGA |
| | | TTCTGAATACGTATTTGCCTAATAGAACAATAAATATACAGGT |
| | | CAGCCTTCGGGCTGGCCTTTTCTTTTAACTATTACCTGTAAC |
| | | ATTTAATTAACAAGTCCAACGTGTTGGACAC |

### SP6

SP6 is a DNA-dependent RNA polymerase first isolated from bacteriophage SP6 infected Salmonella typhimurium. SP6 is structurally similar to T7 and its relatives, but genetically distinct. SP6 is a single-subunit enzyme with high specificity for SP6 promoter sequences. SP6 has proven to be useful in various in vitro contexts and/or reactions, including, but not limited to, large-scale production processes, expression systems, inducible expression systems, RNA editing, and/or RNA interference. SP6, and variants thereof, are commercially available from a variety of vendors (for example, NEB, Promega, Takara Bio, ThermoFisher, Sigma Aldrich).

In some embodiments, a SP6 RNA polymerase useful in accordance with the present invention may be or comprise an N-terminal domain and a polymerase domain, or a fragment thereof. In some embodiments, an amino acid sequence encoding a SP6 RNA polymerase comprises a mutation or variant. In some embodiments, an amino acid sequence encoding a SP6 RNA polymerase comprises a mutation or variant with altered specificity and/or activity relative to an appropriate reference (e.g., a wild-type sequence). In some embodiments, a nucleic acid encoding a SP6 RNA polymerase comprises a codon optimized sequence.

In some embodiments, a SP6 RNA polymerase may be encoded by a homolog or ortholog of a SP6 sequence. In some embodiments, a homolog or ortholog of a SP6 as referred to herein has a sequence homology or identity of at least 80%, at least 85%, at least 90%, or at least 95% with a SP6 sequence (e.g. SEQ ID NO. 71).

SP6 RNA polymerases have been shown to initiate transcription from promoters characterized by a highly conserved nucleic acid sequence (e.g., SEQ ID NO. 73). SP6 RNA polymerases catalyze 5′ to 3′ synthesis of RNA on either ssDNA or dsDNA, using the opposite strand as template, downstream from its promoter. In some embodiments, SP6 RNA polymerase can incorporate modified nucleotides.

In some embodiments, a promoter for use as described herein is 18, 19, 20, 21, 22, or 23 nucleotides in length. In some embodiments, a SP6 promoter nucleotide sequence comprises a variant or mutation which alters binding or recognition by a SP6 RNA polymerase. In some embodiments a SP6 promoter may have a codon optimized nucleic acid sequence.

| SEQ | | |
|---|---|---|
| ID | | |
| NO: | Promoter | Sequence |
| 72 | T7 | TAATACGACTCACTATAG |
| 73 | SP6 | ATTTAGGTGACACTATAG |

### Primers

The present disclosure provides, among other things, an insight that including multiple promoters (e.g., multiple promoters within a single LAMP primer and/or at least one promoter on each of at least two LAMP primers), or no promoter, in certain primer(s) used in templated nucleic acid synthesis (e.g., primer extension), and particularly in LAMP, can provide unexpected improvements.

In some embodiments, a relevant primer includes a sequence complementary to a nucleic acid sequence (e.g., a templated nucleic acid synthesis target nucleic acid). In some embodiments, a primer includes an element that hybridizes to a nucleic acid (i.e., a hybridization element). In some embodiments, a hybridization element has 80%, 85%, 90%, 95%, 99%, sequence identity to a region of a target nucleic acid (e.g., a templated nucleic acid synthesis target nucleic acid).

In some embodiments, a relevant primer or primers includes a promoter sequence element. In some embodiments, a promoter sequence element is an element having the sequence of a promoter. In some embodiments, a promoter sequence element is an element having a sequence complementary to the sequence of a promoter. In some embodiments, a relevant primer or primers includes multiple promoter sequence elements (e.g., multiple promoters within a single LAMP primer and/or at least one promoter on each of at least two LAMP primers). In some embodiments, a primer does not have a promoter sequence or sequences. In some embodiments, a primer includes an element comprising a T7 promoter sequence (i.e., a T7 promoter element). In some embodiments, a T7 promoter element is at the 5′ end of a primer. In some embodiments, a T7 promoter element is at the 3′ end of a primer.

In some embodiments, a primer includes a T7 promoter element and a hybridization element. In some embodiments, a T7 promoter element is located 5′ of a hybridization element. In some embodiments, a T7 promoter element is located 3′ of hybridization element.

In some embodiments, a LAMP reaction utilizes at least 4 distinct primers. In some embodiments, an exemplary LAMP reaction utilizes a forward inner primer (FIP), backward inner primer (BIP), a forward primer (F3), and/or a backward primer (B3). In some embodiments, a LAMP reaction may further utilize loop primers. In some embodiments, loop primers utilized are a forward loop primer (LoopF) and/or a backward loop primer (LoopB) (FIG. 3). In some embodiments, an exemplary LAMP reaction utilizes a F3, a B3, a FIP, a BIP, a LoopF, and/or a LoopB primer. In some embodiments, any one of a F3, a B3, a FIP, a BIP, a FIP, a LoopF and/or a LoopB primer includes a hybridization element, and can optionally further comprise multiple promoters (e.g., multiple promoters within a single LAMP primer and/or at least one promoter on each of at least two LAMP primers).

Templated nucleic acid synthesis reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like.

In some embodiments, a promoter sequence is a sequence that can be used in detection steps. In some embodiments, a primer includes a promoter sequence that can be used with SHERLOCK detection methods. In some embodiments, a primer includes a T7 promoter sequence that can be used with SHERLOCK detection methods.

### CRISPR/Cas Enzymes

In some embodiments, methods and/or compositions of the present disclosure utilize CRISPR/Cas enzymes. In some embodiments, methods and/or compositions of the present disclosure utilize Type V or Type VI CRISPR/Cas enzymes. In some embodiments, methods and/or compositions of the present disclosure utilize Cas12, Cas13, and/or Cas14 CRISPR/Cas enzymes. In some embodiments, methods and/or compositions of the present disclosure utilize CRISPR/Cas enzymes described in WO2016/166340; WO2016/205711; WO/2016/205749; WO2016/205764; WO2017/070605; WO/2017/106657. In some embodiments, methods and/or compositions of the present disclosure utilize Cas13a CRISPR/Cas enzymes. In some embodiments, methods and/or compositions of the present disclosure utilize LwaCas13a CRISPR/Cas enzymes.

In some embodiments, methods and/or compositions of the present disclosure utilize thermostable CRISPR/Cas enzymes. In some embodiments, it will be particularly desirable or useful to utilize a thermostable Cas enzyme. In some embodiments, a useful thermostable Cas protein is a Cas12 or Cas13 homolog (e.g., ortholog). In some embodiments, a thermostable Cas enzyme as described herein may be particularly useful when and/or may permit multiple reaction steps to be performed in a single reaction/vessel (e.g., for “one pot” reactions). Thus, in some embodiments, use of a thermostable Cas may reduce or eliminate certain processing and/or transfer steps. In some embodiments, all reaction steps beyond nucleic acid isolation may be performed in a single vessel (e.g., in a “one-pot” format).

### Uses

Those skilled in the art, reading the present disclosure, will appreciate that provided technologies may be utilized with a variety of contexts involving templated nucleic acid synthesis (e.g., primer extension). For example, primers and/or primer sets as provided herein may be utilized in a templated nucleic acid synthesis reaction, e.g., a LAMP reaction. In some embodiments, provided technologies may be utilized in templated nucleic acid synthesis in a sample, which, for example, may be or comprise a biological or environmental sample. In some embodiments, provided technologies may be used to determine or confirm that a particular target nucleic acid is present and/or quantify how much target nucleic acid is present in a particular sample. In some embodiments methods and/or compositions disclosed herein are directed to technologies for templated nucleic acid synthesis and/or detecting nucleic acids in a sample and/or quantifying how much nucleic acid is present in a particular sample. In some embodiments, templated synthesis is combined with other technologies (e.g., detection technologies). In some embodiments, detection technologies utilize a CRISPR/Cas system, e.g., with collateral activity (e.g., SHERLOCK or DETECTR).

In some embodiments, systems and/or methods provided herein can distinguish even between target nucleic acids that have sequences comprising only a single nucleotide polymorphism(s) (SNPs) to differentiate between said target nucleic acids. In some embodiments, provided technologies can be utilized to detect a SNP-containing nucleic acid. In some embodiments, provided technologies can be utilized to detect SNP-containing nucleic acids in a patient-derived sample or samples. In some embodiments, identification of nucleic acids that have sequences comprising a disease-relevant SNP or disease-relevant SNPs can be utilized for diagnosis and/or informing treatment regimens. In some embodiments, use of multiple guide RNAs in accordance with disclosed technologies may further expand or improve on the number of target nucleic acids that can be distinguished from other target nucleic acids.

In some embodiments, multiple target nucleic acids can be identified simultaneously using disclosed technologies, by employing the use of more than one effector protein, wherein each effector protein targets a nucleic acid with a specific sequence. Multiplex analysis of samples enables large-scale detection of nucleic acids, reducing the time and/or cost of analyses. In accordance with disclosed technologies, alternatives to multiplex analysis may be performed such that multiple effector proteins can be added to a single sample.

In some embodiments, disclosed technologies can achieve detection of one or more microbial or other infectious agents in a sample. In some embodiments, such a sample may be or comprise a biological sample, for example which may have been obtained from a subject, and/or an environmental sample, for example which may be or comprise soil, water, etc. In some embodiments, for example, a microbe may be a bacterium, a fungus, a yeast, a protozoa, a parasite, or a virus.

Disclosed technologies can be used in other methods (or in combination) with other technologies that require identification of a particular microbe species or other infectious agent in a sample or, monitoring the presence of microbe or other infectious agent over time (e.g., by identifying the presence of a particular microbial or infectious proteins (antigens), antibodies, antibody genes, detection of certain phenotypes (e.g., bacterial resistance)), monitoring of disease progression and/or outbreak, and antibiotic screening.

In some embodiments, provided technologies achieve certain benefits and/or advantages, e.g., relative to alternative technologies, for example, such as technologies that may utilize traditional LAMP reactions. For example, in some embodiments, provided technologies may achieve rapid and/or sensitive detection of nucleic acid with a particular sequences, including, in some embodiments, that discriminate between nucleic acids that have sequences comprising only single nucleotide differences. Thus, in some embodiments, provided technologies can identify and/or detect particular microbes or other infectious agents and/or can discriminate between or among different microbes or other infectious agents and/or types of species thereof, even those that may be closely related to one another. In some embodiments, provided technologies can be utilized to identify and/or detect and/or distinguish between microbial species or other infectious agents within a single sample or across multiple samples.

Alternatively or additionally, in some embodiments, provided technologies may be particularly amenable to use in point-of-care devices. Thus, in some embodiments, provided technologies can guide therapeutic regimens (e.g., selection of treatment type and/or dose and/or duration of treatment).

In some embodiments, water samples such as freshwater samples, wastewater samples, or saline water samples can be evaluated for cleanliness and/or safety, and/or potability, to detect the presence of for example, microbial contamination.

### EXEMPLIFICATION

### Example 1: Determination of Optimal T7 Promoter Sequence Design

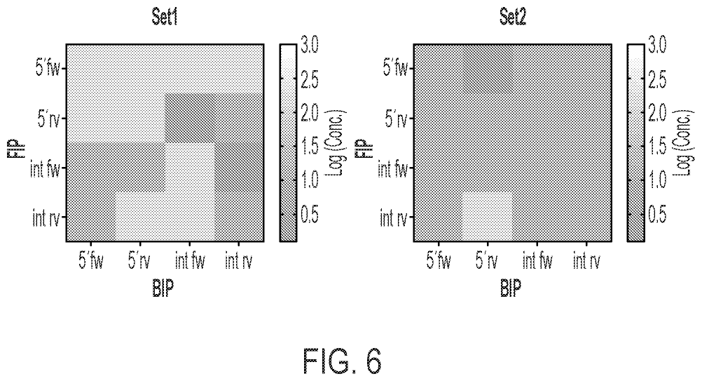

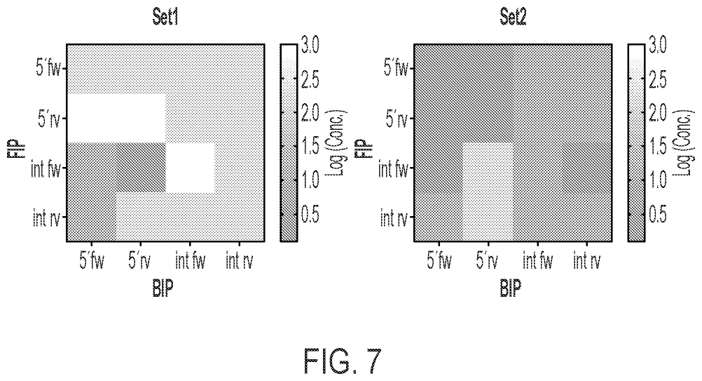

To determine the optimal primer(s) to include a T7 promoter during LAMP, different sets of LAMP primers were tested. Using single-stranded DNA as the template nucleic acid, T7 promoter sequences were added to both the FIP and the BIP with different locations, either inter FIP or the 5′ end, and different directions (forward vs. reverse) (FIG. 5), and tested for SHERLOCK assay performance. SHERLOCK assay performance, using Leptotrichia wadeii (LwaCas13a), is demonstrated as a heat-map where the color indicates the Limit of Detection (LOD) of each assay using certain primer designs. More white-colored results indicate improved performance and more red-colored results indicate reduced performance (FIG. 6). The same primer sets were also tested for SHERLOCK assay performance following SHERLOCK assay targeting at Thermonuclease (FIG. 7). Exemplary results are displayed as a heat-map where the color indicates the Limit of Detection (LOD) of each assay using certain primer designs. More white-colored results indicate improved performance and more red-colored results indicate reduced performance (FIG. 7). Exemplary primers and targets utilized in FIGS. 6 and 7 are shown in Tables 1 and 2, respectively.

TABLE 1

| Seq | | |
|---|---|---|
| ID | | |
| No | Primer | Sequence |
| 1 | ssDNA1 Lamp Set | TTGCTGTTCTACCAAGTAATCCATgaaatTAATACGACTC |
| | 1 FIP (Internal T7 | ACTATAGGGGTAAAACGACGGCCAGTG |
| | forward) | |
| 2 | ssDNA1 Lamp Set | TTGCTGTTCTACCAAGTAATCCATccctatagtgagtcgtattaattt |
| | 1 FIP (Internal T7 | cGTAAAACGACGGCCAGTG |
| | reverse) | |
| 3 | ssDNA1 Lamp Set | gaaatTAATACGACTCACTATAGGGTTGCTGTTCTACCA |
| | 1 FIP (5′ T7 | AGTAATCCATGTAAAACGACGGCCAGTG |
| | forward) | |
| 4 | ssDNA1 Lamp Set | ccctatagtgagtcgtattaatttcTTGCTGTTCTACCAAGTAATCCA |
| | 1 FIP (5′ T7 | TGTAAAACGACGGCCAGTG |
| | reverse) | |
| 5 | ssDNA1 Lamp Set | CGTAATCATGGTCATAGCTGTTTCCgaaatTAATACGACT |
| | 1 BIP (Internal T7 | CACTATAGGGCGGCTCGTATGTTGTGTG |
| | forward) | |
| 6 | ssDNA1 Lamp Set | CGTAATCATGGTCATAGCTGTTTCCccctatagtgagtcgtattaat |
| | 1 BIP (Internal T7 | ttcCGGCTCGTATGTTGTGTG |
| | reverse) | |
| 7 | ssDNA1 Lamp Set | gaaatTAATACGACTCACTATAGGGCGTAATCATGGTCAT |
| | 1 BIP (5′ T7 | AGCTGTTTCCCGGCTCGTATGTTGTGTG |
| | forward) | |
| 8 | ssDNA1 Lamp Set | ccctatagtgagtcgtattaatttcCGTAATCATGGTCATAGCTGTTT |
| | 1 BIP (5′ T7 | CCCGGCTCGTATGTTGTGTG |
| | reverse) | |
| 9 | ssDNA1 Lamp Set | ATTGCTGTTCTACCAAGTAATCCAgaaatTAATACGACTC |
| | 2 FIP (Internal T7 | ACTATAGGGAGTGAATTCGAGCTCGGTA |
| | forward) | |
| 10 | ssDNA1 Lamp Set | ATTGCTGTTCTACCAAGTAATCCAccctatagtgagtcgtattaattt |
| | 2 FIP (Internal T7 | cAGTGAATTCGAGCTCGGTA |
| | reverse) | |
| 11 | ssDNA1 Lamp Set | gaaatTAATACGACTCACTATAGGGATTGCTGTTCTACCA |
| | 2 FIP (5′ T7 | AGTAATCCAAGTGAATTCGAGCTCGGTA |
| | forward) | |
| 12 | ssDNA1 Lamp Set | ccctatagtgagtcgtattaatttcATTGCTGTTCTACCAAGTAATCC |
| | 2 FIP (5′ T7 | AAGTGAATTCGAGCTCGGTA |
| | reverse) | |
| 13 | ssDNA1 Lamp Set | CGTAATCATGGTCATAGCTGTTTCCgaaatTAATACGACT |
| | 2 BIP (Internal T7 | CACTATAGGGCGGCTCGTATGTTGTGTG |
| | forward) | |
| 14 | ssDNA1 Lamp Set | CGTAATCATGGTCATAGCTGTTTCCccctatagtgagtcgtattaat |
| | 2 BIP (Internal T7 | ttcCGGCTCGTATGTTGTGTG |
| | reverse) | |
| 15 | ssDNA1 Lamp Set | gaaatTAATACGACTCACTATAGGGCGTAATCATGGTCAT |
| | 2 BIP (5′ T7 | AGCTGTTTCCCGGCTCGTATGTTGTGTG |
| | forward) | |
| 16 | ssDNA1 Lamp Set | ccctatagtgagtcgtattaatttcCGTAATCATGGTCATAGCTGTTT |
| | 2 BIP (5′ T7 | CCCGGCTCGTATGTTGTGTG |
| | reverse) | |
| 17 | LAMP BIP | CGTAATCATGGTCATAGCTGTTTCCCGGCTCGTATGTT |
| | ssDNA1 1 | GTGTG |
| 18 | LAMP F3 | CAGGGTTTTCCCAGTCAC |
| | SSDNA1 1 | |
| 19 | LAMP B3 | AGGCTTTACACTTTATGCTTC |
| | SSDNA1 1 | |
| 20 | LAMP LF | GGGTACCGAGCTCGAATT |
| | SSDNA1 1 | |
| 21 | LAMP LB | TGTGTTTATCCGCTCACAATTC |
| | SSDNA1 1 | |
| 22 | LAMP BIP | CGTAATCATGGTCATAGCTGTTTCCCGGCTCGTATGTT |
| | SSDNA1 2 | GTGTG |
| 23 | LAMP F3 | GTCACGACGTTGTAAAACG |
| | SSDNA1 2 | |
| 24 | LAMP B3 | AGGCTTTACACTTTATGCTTC |
| | SSDNA1 2 | |
| 25 | LAMP LF | ATTTCTAGAGGATCCCCGG |
| | SSDNA1 2 | |
| 26 | LAMP LB | TGTGTTTATCCGCTCACAATTC |
| | SSDNA1 2 | |
| 27 | ssDNA1 target | CGCCAGCTGGCGAAAGGGGGATGTGCTGCAAGGCGA |
| | | TTAAGTTGGGTAACGCCAGGGTTTTCCCAGTCACGAC |
| | | GTTGTAAAACGACGGCCAGTGAATTCGAGCTCGGTA |
| | | CCCGGGGATCCTCTAGAAATATGGATTACTTGgtAGAA |
| | | CAGCAATCTACTCGACCTGCAGGCATGCAAGCTTGGC |
| | | GTAATCATGGTCATAGCTGTTTCCTGTGTTTATCCGCT |
| | | CACAATTCCACACAACATACGAGCCGGAAGCATAAA |
| | | GTGTAAAGCCTGGGGTGCCTAATGAGTGAGCTAACTC |
| | | ACATTAATTGCGTTGCGCTCACTGCCCGCTTTCCAGT |
| | | CGGGAAACCTGTCGTGCCAGCTGCATTAATGAATCGG |
| | | CCAACGCGC |

TABLE 2

| Seq | | |
|---|---|---|
| ID | | |
| NO | Primer | Sequence |
| 28 | Thermonuclease | CGCTTTAATTAATGTCGCAGGTTCTgaaatTAATACGAC |
| | Lamp Set 1 FIP | TCACTATAGGGTAGAAGTGGTTCTGAAGATCC |
| | (Internal T7 | |
| | forward) | |
| 29 | Thermonuclease | CGCTTTAATTAATGTCGCAGGTTCTccctatagtgagtcgtattaa |
| | Lamp Set 1 FIP | tttcTAGAAGTGGTTCTGAAGATCC |
| | (Internal T7 | |
| | reverse) | |
| 30 | Thermonuclease | gaaatTAATACGACTCACTATAGGGCGCTTTAATTAATG |
| | Lamp Set 1 FIP (5′ | TCGCAGGTTCTTAGAAGTGGTTCTGAAGATCC |
| | T7 forward) | |
| 31 | Thermonuclease | ccctatagtgagtcgtattaatttcCGCTTTAATTAATGTCGCAGGTT |
| | Lamp Set 1 FIP (5′ | CTTAGAAGTGGTTCTGAAGATCC |
| | T7 reverse) | |
| 32 | Thermonuclease | AATGTACAAAGGTCAACCAATGACAgaaatTAATACGA |
| | Lamp Set 1 BIP | CTCACTATAGGGGGATGCTTTGTTTCAGGTG |
| | (Internal T7 | |
| | forward) | |
| 33 | Thermonuclease | AATGTACAAAGGTCAACCAATGACAccctatagtgagtcgtatt |
| | Lamp Set 1 BIP | aatttcGGATGCTTTGTTTCAGGTG |
| | (Internal T7 | |
| | reverse) | |
| 34 | Thermonuclease | gaaatTAATACGACTCACTATAGGGAATGTACAAAGGTC |
| | Lamp Set 1 BIP (5′ | AACCAATGACAGGATGCTTTGTTTCAGGTG |
| | T7 forward) | |
| 35 | Thermonuclease | ccctatagtgagtcgtattaatttcAATGTACAAAGGTCAACCAATG |
| | Lamp Set 1 BIP (5′ | ACAGGATGCTTTGTTTCAGGTG |
| | T7 reverse) | |
| 36 | Thermonuclease | TGTCATTGGTTGACCTTTGTACATTgaaatTAATACGAC |
| | Lamp Set 2 FIP | TCACTATAGGGAAAATTACATAAAGAACCTGCGA |
| | (Internal T7 | |
| | forward) | |
| 37 | Thermonuclease | TGTCATTGGTTGACCTTTGTACATTccctatagtgagtcgtattaa |
| | Lamp Set 2 FIP | tttcAAAATTACATAAAGAACCTGCGA |
| | (Internal T7 | |
| | reverse) | |
| 38 | Thermonuclease | gaaatTAATACGACTCACTATAGGGTGTCATTGGTTGAC |
| | Lamp Set 2 FIP (5′ | CTTTGTACATTAAAATTACATAAAGAACCTGCGA |
| | T7 forward) | |
| 39 | Thermonuclease | ccctatagtgagtcgtattaatttcTGTCATTGGTTGACCTTTGTAC |
| | Lamp Set 2 FIP (5′ | ATTAAAATTACATAAAGAACCTGCGA |
| | T7 reverse) | |
| 40 | Thermonuclease | GTTGATACACCTGAAACAAAGCATCgaaatTAATACGA |
| | Lamp Set 2 BIP | CTCACTATAGGGATCTTTTTCGTAAATGCACTTG |
| | (Internal T7 | |
| | forward) | |
| 41 | Thermonuclease | GTTGATACACCTGAAACAAAGCATCccctatagtgagtcgtatt |
| | Lamp Set 2 BIP | aatttcATCTTTTTCGTAAATGCACTTG |
| | (Internal T7 | |
| | reverse) | |
| 42 | Thermonuclease | gaaatTAATACGACTCACTATAGGGGTTGATACACCTGA |
| | Lamp Set 2 BIP (5′ | AACAAAGCATCATCTTTTTCGTAAATGCACTTG |
| | T7 forward) | |
| 43 | Thermonuclease | ccctatagtgagtcgtattaatttcGTTGATACACCTGAAACAAAGC |
| | Lamp Set 2 BIP (5′ | ATCATCTTTTTCGTAAATGCACTTG |
| | T7 reverse) | |
| 44 | LAMP BIP | AATGTACAAAGGTCAACCAATGACAGGATGCTTTGT |
| | Thermo 1 | TTCAGGTG |
| 45 | LAMP F3 Thermo | CACAAACAGATAATGGCGTAA |
| | 1 | |
| 46 | LAMP B3 Thermo | AGGACCATATTTCTCTACACC |
| | 1 | |
| 47 | LAMP LF Thermo | TGAAGTTGCACTATATACTGTT |
| | 1 | |
| 48 | LAMP LB Thermo | NONE |
| | 1 | |
| 49 | LAMP BIP | GTTGATACACCTGAAACAAAGCATCATCTTTTTCGTA |
| | Thermo 2 | AATGCACTTG |
| 50 | LAMP F3 Thermo | AACAGTATATAGTGCAACTTCAA |
| | 2 | |
| 51 | LAMP B3 Thermo | CTTTGTCAAACTCGACTTCAA |
| | 2 | |
| 52 | LAMP LF Thermo | CAGTATCACCATCAATCGCTTT |
| | 2 | |
| 53 | LAMP LB Thermo | AAAGGTGTAGAGAAATATGGTCCTG |
| | 2 | |
| 54 | Thermonuclease | TTAGTGTTAACTTTAGTTGTAGCTTCAAGTCTAAGTA |
| | target | GCTCAGCAAATGCATCACAAACAGATAATGGCGTAA |
| | | ATAGAAGTGGTTCTGAAGATCCAACAGTATATAGTGC |
| | | AACTTCAACTAAAAAATTACATAAAGAACCTGCGAC |
| | | ATTAATTAAAGCGATTGATGGTGATACTGTTAAATTAA |
| | | TGTACAAAGGTCAACCAATGACATTCAGACTATTATT |
| | | GGTTGATACACCTGAAACAAAGCATCCTAAAAAAGG |
| | | TGTAGAGAAATATGGTCCTGAAGCAAGTGCATTTAC |
| | | GAAAAAGATGGTAGAAAATGCAAAGAAAATTGAAG |
| | | TCGAGTTTGACAAAGGTCAAAGAACTGATAAATATG |
| | | GACGTGGCTTAGCGTATATTTATGCTGATGGAAAAAT |
| | | GGTAAACGAAGCTTTAGTTCGTCAAGGCTTGGCTAA |
| | | AGTTGCTTATGTTTATAAACCTAAC |

### Example 2: Inclusion of T7 Promoter on Loop Primers Enhances LAMP Reaction Speed

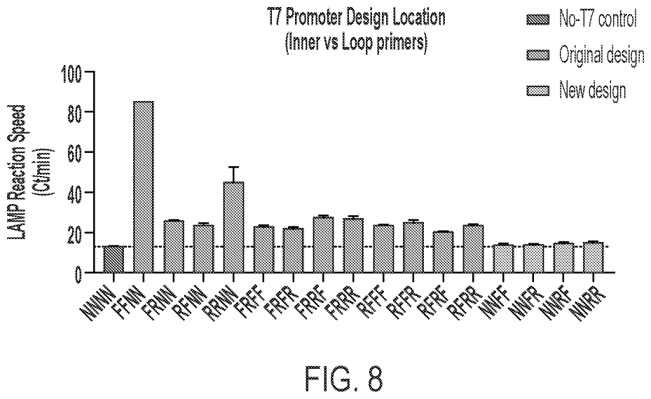

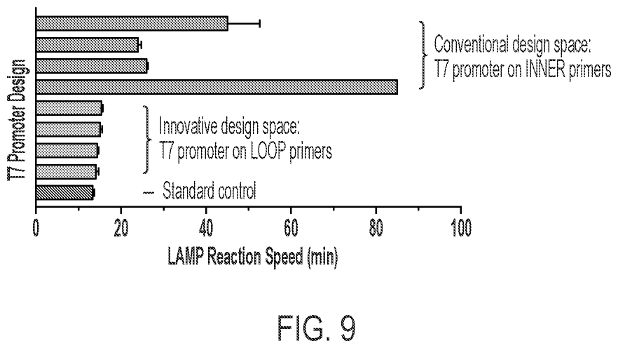

To determine the effect of T7 promoter inclusion in inner primers verse loop primers (no T7), LAMP reaction speed for amplification of dsDNA was measured and compared to no T7 promoter control primers. Detection was completed using Sybr Green. Inclusion of a T7 promoter sequence on loop primers can increase LAMP reaction speed (FIG. 8-9). Table 3 provides primer sequences utilized in this example.

TABLE 3

| Seq | | |
|---|---|---|
| ID | | |
| NO | Primer | Sequence |
| 55 | FluA FIP- | CACTTTCCAGTCTCTGCGCGccctatagtgagtcgt |
| | T7 reverse | attaGTACGTTCTTTCTATCATCCCG |
| 56 | FluA BIP- | CTCTCATGGAATGGCTAAAGACccctatagtgagtc |
| | T7 reverse | gtattaAAGCACGGTGAGCGTGAAC |
| 57 | FluA LF- | ccctatagtgagtcgtattaGCTTTGAGGGGGCCTG |
| | T7 reverse | A |
| 58 | FluA LB- | ccctatagtgagtcgtattaACCAATCTTGTCACCT |
| | T7 reverse | CTGACT |
| 59 | FluA FIP- | CACTTTCCAGTCTCTGCGCGgaaatTAATACGACTC |
| | T7 forward | ACTATAGGGGTACGTTCTTTCTATCATCCCG |
| 60 | FluA BIP- | CTCTCATGGAATGGCTAAAGACgaaatTAATACGAC |
| | T7 forward | TCACTATAGGGAAGCACGGTGAGCGTGAAC |
| 61 | FluA LF- | gaaatTAATACGACTCACTATAGGGGCTTTGAGGGG |
| | T7 forward | GCCTGA |
| 62 | FluA LB- | gaaatTAATACGACTCACTATAGGGACCAATCTTGT |
| | T7 forward | CACCTCTGACT |
| 63 | FluA F3 | TCTAACCGAGGTCGAAAC |
| 64 | FluA B3 | TCTACGCTGCAGTCCTC |
| 65 | FluA FIP | CACTTTCCAGTCTCTGCGCGGTACGTTCTTTCTATC |
| | | ATCCCG |
| 66 | FluA BIP | CTCTCATGGAATGGCTAAAGACAAGCACGGTGAGCG |
| | | TGAAC |
| 67 | FluA LF | GCTTTGAGGGGGCCTGA |
| 68 | FluA LB | ACCAATCTTGTCACCTCTGACT |

### Example 3: T7 Promoter can Improve Cas13a Detection

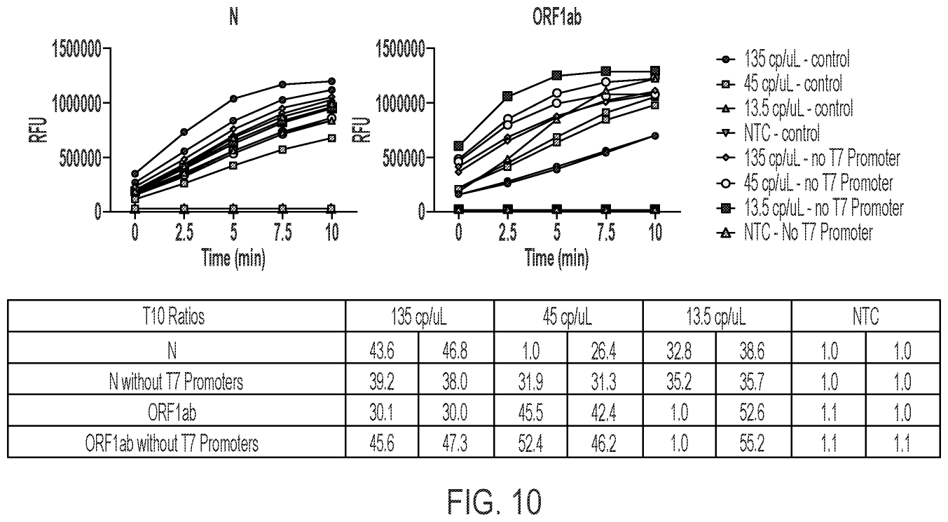

To determine whether T7 promoters improve Cas13a detection following LAMP amplification, N and ORFlab of SARS-COV-2 were LAMP-amplified. Amplification was detected by a Cas13a-based SHERLOCK detection method. Amplification primers were either standard loop primers that contained a single T7 promoter sequence in a forward loop or loop primers without a T7 promoter sequence (no T7 promoter, control). Varying concentrations of template were added to the reaction mixture and no template (NTC) was used as a negative control. LAMP-amplification completed with primers without a T7 promoter sequence was detected by Cas13a-SHERLOCK to comparable levels of amplification completed with primers including a T7 promoter sequence in one loop, suggesting T7 promoters are not required for Cas13a-based detection post-LAMP, but can improve detection of nucleic acids (FIG. 10).

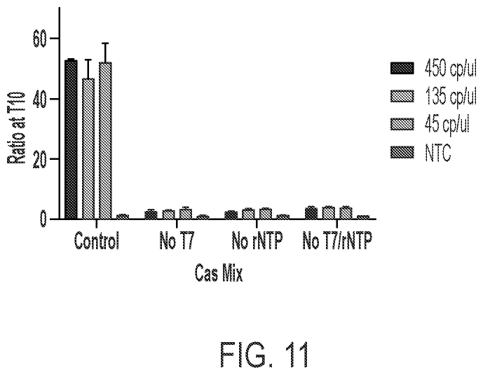

T7 polymerase and rNTPs are required for Cas13a-based detection post-LAMP (FIG. 11). To determine T7 polymerase and rNTPs are required for Cas13a-based detection post-LAMP, LAMP amplification for Orflab was conducted according to the manufacturer's instructions in the SHERLOCK Integrated DNA Technologies (IDT) Kit. No T7 promoters are present in the IDT Kit LAMP primers. Following LAMP-amplification, in the presence or absence of either 1 unit/μL T7 polymerase and/or 1 mM rNTPs, Cas13a-based detection was used. NTC was used as a negative control. LAMP-amplification was detected (Relative Fluorescence Units, RFU) after 10 minutes. Data are presented as a ratio of measured RFU at 10 minutes of a reaction divided by RFU at 10 minutes of a NTC reaction. LAMP reactions that included both T7 and rNTPs increased signal approximately 50-fold compared to NTC. One of the two replicates of ‘45 cp/ul control’ failed to produce a positive signal (ratio of 1.0) and as such, data are represented as are two distinct traces of pink squares. LAMP reactions that did not include T7 and/or rNTPs showed no signal increase compared to NTC, suggesting both T7 and rNTPs are required for Cas13a-based detection post-LAMP (FIG. 11).

### EQUIVALENTS

Those skilled in the art will recognize, or be able to ascertain using no more than routine experimentation, many equivalents to the specific embodiments of the invention described herein. The scope of the systems and methods provided herein is not intended to be limited to the above Description, but rather is as set forth in the following claims:

1. A method comprising:
i. contacting a templated nucleic acid synthesis target with:
(a) a plurality of primers, wherein:
(1) the plurality includes at least one set of forward and reverse primers, each of which includes a templated nucleic acid synthesis target hybridization element selected so that the primers of the pair bind to opposite strands of a templated nucleic acid synthesis target, flanking a target sequence of interest;
(2) the plurality of primers furthermore includes at least two T7 promoter sequence elements; and
(b) amplification reagents;
ii. incubating the templated nucleic acid synthesis target, plurality of primers; and
amplification reagents so an amplified nucleic acid comprising the target sequence of interest and the at least two T7 promoter sequence elements is generated;
iii. contacting the amplified nucleic acid comprising the target sequence of interest with a CRISPR-Cas detection composition; and
iv. detecting the amplified nucleic acid.

2. The method of claim 1, wherein each templated nucleic acid synthesis target hybridization element is at least 80% complementary to its hybridization site in the templated nucleic acid synthesis target nucleic acid.

3-4. (canceled)

5. The method of claim 1, wherein the method further comprises a step of:
isolating the templated nucleic acid synthesis target from a sample.

6-15. (canceled)

16. The method of claim 1, wherein the plurality of primers comprises at least one primer including the at least two T7 promoter sequence elements.

17. The method of claim 1, wherein the plurality of primers comprises at least two primers that each include at least one of the at least two T7 promoter sequence elements.

18. (canceled)

19. The method of claim 17, wherein both the forward and backward loop primers comprise a T7 promoter sequence element.

20. The method of claim 17, wherein one or more of forward inner primer (FIP); backward inner primer (BIP); forward outer primer (F3); and/or backward outer primer (B3) comprise a T7 promoter element.

21. The method of claim 17, wherein one or more of a forward inner primer (FIP); backward inner primer (BIP); a forward outer primer (F3); a backward outer primer (B3); a forward loop primer (LoopF); and/or a backward loop primer (LoopB) comprise a T7 promoter element.

22. The method of claim 1, wherein the amplification is a polymerase chain reaction amplification or an isothermal amplification reaction.

23. (canceled)

24. The method of claim 22, wherein the isothermal amplification reaction is loop-mediated isothermal amplification (LAMP).

25. The method of claim 1, wherein the amplified nucleic acid is detected via a fluorescence, absorbance, spectrometry, lateral flow, migration, chemiluminescence, or electrochemical methods.

26. (canceled)

27. The method of claim 1, wherein the CRISPR-Cas detection composition comprises:
(i) a guide polynucleotide capable of binding the target sequence of interest;
(ii) a labeled nucleic acid reporter construct; and
(iii) at least one Cas protein.

28. The method of claim 27, wherein the method comprises transcribing a copied and/or amplified templated nucleic acid synthesis target using any primer inserted promoter.

29. The method of claim 28, wherein the Cas protein is Cas13 or Cas12.

30. (canceled)

31. The method of claim 28, wherein the Cas protein is Cas13a or Cas13b.

32. (canceled)

33. The method of claim 27, wherein the Cas is a thermostable Cas.

34. The method of claim 27, wherein the Cas detection system comprises more than one Cas protein.

35. The method of claim 34, wherein the more than one Cas protein comprises Cas 13 and Cas12.

36. The method of claim 1, wherein the templated nucleic acid synthesis target comprises at least two target sequences of interest.

37. The method of claim 36, wherein the pair of primers bind to the templated nucleic acid synthesis target so that they flank the at least two target sequences of interest.

38. (canceled)

39. A composition comprising:
(a) a plurality of primers, wherein the plurality includes at least one set of forward and reverse primers, each of which includes a templated nucleic acid synthesis target hybridization element selected so that at least one forward primer and at least one reverse primer bind to opposite strands of a template nucleic acid synthesis target, flanking a target sequence of interest; wherein the plurality of primers furthermore includes at least two T7 promoter sequence elements and each templated nucleic acid synthesis target hybridization element is at least 80% complementary to its hybridization site in the template nucleic acid synthesis target;
(b) amplification reagents; and
(c) a CRISPR-Cas detection composition.

40. The composition of claim 39, wherein the T7 promoter sequence element is located at the 3′ or 5′ end of at least one primer.

41. The composition of claim 39, wherein the T7 promoter sequence element is located 3′ or 5′ of the hybridization element.

42. The composition of claim 39, wherein one or more of a forward loop primer (LoopF); and/or a backward loop primer (LoopB) comprise a T7 promoter element.

43. The composition of claim 39, wherein one or more of a forward inner primer (FIP); backward inner primer (BIP); a forward outer primer (F3); a backward outer primer (B3); a forward loop primer (LoopF); and/or a backward loop primer (LoopB) comprise a T7 promoter element.

44-75. (canceled)


SEQUENCE LISTING (73 sequences)

SEQ ID NO: 1 - DNA, 67 bp, Artificial Sequence
  source: /note=“Description of Artificial Sequence: Synthetic primer”
ttgctgttctaccaagtaatccatgaaattaatacgactcactataggggtaaaacgacggccagtg

SEQ ID NO: 2 - DNA, 67 bp, Artificial Sequence
  source: /note=“Description of Artificial Sequence: Synthetic primer”
ttgctgttctaccaagtaatccatccctatagtgagtcgtattaatttcgtaaaacgacggccagtg

SEQ ID NO: 3 - DNA, 67 bp, Artificial Sequence
  source: /note=“Description of Artificial Sequence: Synthetic primer”
gaaattaatacgactcactatagggttgctgttctaccaagtaatccatgtaaaacgacggccagtg

SEQ ID NO: 4 - DNA, 67 bp, Artificial Sequence
  source: /note=“Description of Artificial Sequence: Synthetic primer”
ccctatagtgagtcgtattaatttcttgctgttctaccaagtaatccatgtaaaacgacggccagtg

SEQ ID NO: 5 - DNA, 68 bp, Artificial Sequence
  source: /note=“Description of Artificial Sequence: Synthetic primer”
cgtaatcatggtcatagctgtttccgaaattaatacgactcactatagggcggctcgtatgttgtgtg

SEQ ID NO: 6 - DNA, 68 bp, Artificial Sequence
  source: /note=“Description of Artificial Sequence: Synthetic primer”
cgtaatcatggtcatagctgtttccccctatagtgagtcgtattaatttccggctcgtatgttgtgtg

SEQ ID NO: 7 - DNA, 68 bp, Artificial Sequence
  source: /note=“Description of Artificial Sequence: Synthetic primer”
gaaattaatacgactcactatagggcgtaatcatggtcatagctgtttcccggctcgtatgttgtgtg

SEQ ID NO: 8 - DNA, 68 bp, Artificial Sequence
  source: /note=“Description of Artificial Sequence: Synthetic primer”
ccctatagtgagtcgtattaatttccgtaatcatggtcatagctgtttcccggctcgtatgttgtgtg

SEQ ID NO: 9 - DNA, 68 bp, Artificial Sequence
  source: /note=“Description of Artificial Sequence: Synthetic primer”
attgctgttctaccaagtaatccagaaattaatacgactcactatagggagtgaattcgagctcggta

SEQ ID NO: 10 - DNA, 68 bp, Artificial Sequence
  source: /note=“Description of Artificial Sequence: Synthetic primer”
attgctgttctaccaagtaatccaccctatagtgagtcgtattaatttcagtgaattcgagctcggta

SEQ ID NO: 11 - DNA, 68 bp, Artificial Sequence
  source: /note=“Description of Artificial Sequence: Synthetic primer”
gaaattaatacgactcactatagggattgctgttctaccaagtaatccaagtgaattcgagctcggta

SEQ ID NO: 12 - DNA, 68 bp, Artificial Sequence
  source: /note=“Description of Artificial Sequence: Synthetic primer”
ccctatagtgagtcgtattaatttcattgctgttctaccaagtaatccaagtgaattcgagctcggta

SEQ ID NO: 13 - DNA, 68 bp, Artificial Sequence; identical to SEQ ID NO: 5
  source: /note=“Description of Artificial Sequence: Synthetic primer”

SEQ ID NO: 14 - DNA, 68 bp, Artificial Sequence; identical to SEQ ID NO: 6
  source: /note=“Description of Artificial Sequence: Synthetic primer”

SEQ ID NO: 15 - DNA, 68 bp, Artificial Sequence; identical to SEQ ID NO: 7
  source: /note=“Description of Artificial Sequence: Synthetic primer”

SEQ ID NO: 16 - DNA, 68 bp, Artificial Sequence; identical to SEQ ID NO: 8
  source: /note=“Description of Artificial Sequence: Synthetic primer”

SEQ ID NO: 17 - DNA, 43 bp, Artificial Sequence
  source: /note=“Description of Artificial Sequence: Synthetic primer”
cgtaatcatggtcatagctgtttcccggctcgtatgttgtgtg

SEQ ID NO: 18 - DNA, 18 bp, Artificial Sequence
  source: /note=“Description of Artificial Sequence: Synthetic primer”
cagggttttcccagtcac

SEQ ID NO: 19 - DNA, 21 bp, Artificial Sequence
  source: /note=“Description of Artificial Sequence: Synthetic primer”
aggctttacactttatgcttc

SEQ ID NO: 20 - DNA, 18 bp, Artificial Sequence
  source: /note=“Description of Artificial Sequence: Synthetic primer”
gggtaccgagctcgaatt

SEQ ID NO: 21 - DNA, 22 bp, Artificial Sequence
  source: /note=“Description of Artificial Sequence: Synthetic primer”
tgtgtttatccgctcacaattc

SEQ ID NO: 22 - DNA, 43 bp, Artificial Sequence; identical to SEQ ID NO: 17
  source: /note=“Description of Artificial Sequence: Synthetic primer”

SEQ ID NO: 23 - DNA, 19 bp, Artificial Sequence
  source: /note=“Description of Artificial Sequence: Synthetic primer”
gtcacgacgttgtaaaacg

SEQ ID NO: 24 - DNA, 21 bp, Artificial Sequence
  source: /note=“Description of Artificial Sequence: Synthetic primer”
aggctttacactttatgcttc

SEQ ID NO: 25 - DNA, 19 bp, Artificial Sequence
  source: /note=“Description of Artificial Sequence: Synthetic primer”
atttctagaggatccccgg

SEQ ID NO: 26 - DNA, 22 bp, Artificial Sequence
  source: /note=“Description of Artificial Sequence: Synthetic primer”
tgtgtttatccgctcacaattc

SEQ ID NO: 27 - DNA, 378 bp, Artificial Sequence
  source: /note=“Description of Artificial Sequence: Synthetic primer”
cgccagctggcgaaagggggatgtgctgcaaggcgattaagttgggtaacgccagggttttcccagtcacgacgttgtaaaacgacggccagtgaattcgagctcggtacccggggatcctctagaaatatggattacttggtagaacagcaatctactcgacctgcaggcatgcaagcttggcgtaatcatggtcatagctgtttcctgtgtttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgc

SEQ ID NO: 28 - DNA, 71 bp, Artificial Sequence
  source: /note=“Description of Artificial Sequence: Synthetic primer”
cgctttaattaatgtcgcaggttctgaaattaatacgactcactatagggtagaagtggttctgaagatcc

SEQ ID NO: 29 - DNA, 71 bp, Artificial Sequence
  source: /note=“Description of Artificial Sequence: Synthetic primer”
cgctttaattaatgtcgcaggttctccctatagtgagtcgtattaatttctagaagtggttctgaagatcc

SEQ ID NO: 30 - DNA, 71 bp, Artificial Sequence
  source: /note=“Description of Artificial Sequence: Synthetic primer”
gaaattaatacgactcactatagggcgctttaattaatgtcgcaggttcttagaagtggttctgaagatcc

SEQ ID NO: 31 - DNA, 71 bp, Artificial Sequence
  source: /note=“Description of Artificial Sequence: Synthetic primer”
ccctatagtgagtcgtattaatttccgctttaattaatgtcgcaggttcttagaagtggttctgaagatcc

SEQ ID NO: 32 - DNA, 69 bp, Artificial Sequence
  source: /note=“Description of Artificial Sequence: Synthetic primer”
aatgtacaaaggtcaaccaatgacagaaattaatacgactcactatagggggatgctttgtttcaggtg

SEQ ID NO: 33 - DNA, 69 bp, Artificial Sequence
  source: /note=“Description of Artificial Sequence: Synthetic primer”
aatgtacaaaggtcaaccaatgacaccctatagtgagtcgtattaatttcggatgctttgtttcaggtg

SEQ ID NO: 34 - DNA, 69 bp, Artificial Sequence
  source: /note=“Description of Artificial Sequence: Synthetic primer”
gaaattaatacgactcactatagggaatgtacaaaggtcaaccaatgacaggatgctttgtttcaggtg

SEQ ID NO: 35 - DNA, 69 bp, Artificial Sequence
  source: /note=“Description of Artificial Sequence: Synthetic primer”
ccctatagtgagtcgtattaatttcaatgtacaaaggtcaaccaatgacaggatgctttgtttcaggtg

SEQ ID NO: 36 - DNA, 73 bp, Artificial Sequence
  source: /note=“Description of Artificial Sequence: Synthetic primer”
tgtcattggttgacctttgtacattgaaattaatacgactcactatagggaaaattacataaagaacctgcga

SEQ ID NO: 37 - DNA, 73 bp, Artificial Sequence
  source: /note=“Description of Artificial Sequence: Synthetic primer”
tgtcattggttgacctttgtacattccctatagtgagtcgtattaatttcaaaattacataaagaacctgcga

SEQ ID NO: 38 - DNA, 73 bp, Artificial Sequence
  source: /note=“Description of Artificial Sequence: Synthetic primer”
gaaattaatacgactcactatagggtgtcattggttgacctttgtacattaaaattacataaagaacctgcga

SEQ ID NO: 39 - DNA, 73 bp, Artificial Sequence
  source: /note=“Description of Artificial Sequence: Synthetic primer”
ccctatagtgagtcgtattaatttctgtcattggttgacctttgtacattaaaattacataaagaacctgcga

SEQ ID NO: 40 - DNA, 72 bp, Artificial Sequence
  source: /note=“Description of Artificial Sequence: Synthetic primer”
gttgatacacctgaaacaaagcatcgaaattaatacgactcactatagggatctttttcgtaaatgcacttg

SEQ ID NO: 41 - DNA, 72 bp, Artificial Sequence
  source: /note=“Description of Artificial Sequence: Synthetic primer”
gttgatacacctgaaacaaagcatcccctatagtgagtcgtattaatttcatctttttcgtaaatgcacttg

SEQ ID NO: 42 - DNA, 72 bp, Artificial Sequence
  source: /note=“Description of Artificial Sequence: Synthetic primer”
gaaattaatacgactcactataggggttgatacacctgaaacaaagcatcatctttttcgtaaatgcacttg

SEQ ID NO: 43 - DNA, 72 bp, Artificial Sequence
  source: /note=“Description of Artificial Sequence: Synthetic primer”
ccctatagtgagtcgtattaatttcgttgatacacctgaaacaaagcatcatctttttcgtaaatgcacttg

SEQ ID NO: 44 - DNA, 44 bp, Artificial Sequence
  source: /note=“Description of Artificial Sequence: Synthetic primer”
aatgtacaaaggtcaaccaatgacaggatgctttgtttcaggtg

SEQ ID NO: 45 - DNA, 21 bp, Artificial Sequence
  source: /note=“Description of Artificial Sequence: Synthetic primer”
cacaaacagataatggcgtaa

SEQ ID NO: 46 - DNA, 21 bp, Artificial Sequence
  source: /note=“Description of Artificial Sequence: Synthetic primer”
aggaccatatttctctacacc

SEQ ID NO: 47 - DNA, 22 bp, Artificial Sequence
  source: /note=“Description of Artificial Sequence: Synthetic primer”
tgaagttgcactatatactgtt

SEQ ID NO: 48 - skipped

SEQ ID NO: 49 - DNA, 47 bp, Artificial Sequence
  source: /note=“Description of Artificial Sequence: Synthetic primer”
gttgatacacctgaaacaaagcatcatctttttcgtaaatgcacttg

SEQ ID NO: 50 - DNA, 23 bp, Artificial Sequence
  source: /note=“Description of Artificial Sequence: Synthetic primer”
aacagtatatagtgcaacttcaa

SEQ ID NO: 51 - DNA, 21 bp, Artificial Sequence
  source: /note=“Description of Artificial Sequence: Synthetic primer”
ctttgtcaaactcgacttcaa

SEQ ID NO: 52 - DNA, 22 bp, Artificial Sequence
  source: /note=“Description of Artificial Sequence: Synthetic primer”
cagtatcaccatcaatcgcttt

SEQ ID NO: 53 - DNA, 25 bp, Artificial Sequence
  source: /note=“Description of Artificial Sequence: Synthetic primer”
aaaggtgtagagaaatatggtcctg

SEQ ID NO: 54 - DNA, 462 bp, Artificial Sequence
  source: /note=“Description of Artificial Sequence: Synthetic primer”
ttagtgttaactttagttgtagcttcaagtctaagtagctcagcaaatgcatcacaaacagataatggcgtaaatagaagtggttctgaagatccaacagtatatagtgcaacttcaactaaaaaattacataaagaacctgcgacattaattaaagcgattgatggtgatactgttaaattaatgtacaaaggtcaaccaatgacattcagactattattggttgatacacctgaaacaaagcatcctaaaaaaggtgtagagaaatatggtcctgaagcaagtgcatttacgaaaaagatggtagaaaatgcaaagaaaattgaagtcgagtttgacaaaggtcaaagaactgataaatatggacgtggcttagcgtatatttatgctgatggaaaaatggtaaacgaagctttagttcgtcaaggcttggctaaagttgcttatgtttataaacctaac

SEQ ID NO: 55 - DNA, 62 bp, Artificial Sequence
  source: /note=“Description of Artificial Sequence: Synthetic primer”
cactttccagtctctgcgcgccctatagtgagtcgtattagtacgttctttctatcatcccg

SEQ ID NO: 56 - DNA, 61 bp, Artificial Sequence
  source: /note=“Description of Artificial Sequence: Synthetic primer”
ctctcatggaatggctaaagacccctatagtgagtcgtattaaagcacggtgagcgtgaac

SEQ ID NO: 57 - DNA, 37 bp, Artificial Sequence
  source: /note=“Description of Artificial Sequence: Synthetic primer”
ccctatagtgagtcgtattagctttgagggggcctga

SEQ ID NO: 58 - DNA, 42 bp, Artificial Sequence
  source: /note=“Description of Artificial Sequence: Synthetic primer”
ccctatagtgagtcgtattaaccaatcttgtcacctctgact

SEQ ID NO: 59 - DNA, 67 bp, Artificial Sequence
  source: /note=“Description of Artificial Sequence: Synthetic primer”
cactttccagtctctgcgcggaaattaatacgactcactataggggtacgttctttctatcatcccg

SEQ ID NO: 60 - DNA, 66 bp, Artificial Sequence
  source: /note=“Description of Artificial Sequence: Synthetic primer”
ctctcatggaatggctaaagacgaaattaatacgactcactatagggaagcacggtgagcgtgaac

SEQ ID NO: 61 - DNA, 42 bp, Artificial Sequence
  source: /note=“Description of Artificial Sequence: Synthetic primer”
gaaattaatacgactcactataggggctttgagggggcctga

SEQ ID NO: 62 - DNA, 47 bp, Artificial Sequence
  source: /note=“Description of Artificial Sequence: Synthetic primer”
gaaattaatacgactcactatagggaccaatcttgtcacctctgact

SEQ ID NO: 63 - DNA, 18 bp, Artificial Sequence
  source: /note=“Description of Artificial Sequence: Synthetic primer”
tctaaccgaggtcgaaac

SEQ ID NO: 64 - DNA, 17 bp, Artificial Sequence
  source: /note=“Description of Artificial Sequence: Synthetic primer”
tctacgctgcagtcctc

SEQ ID NO: 65 - DNA, 42 bp, Artificial Sequence
  source: /note=“Description of Artificial Sequence: Synthetic primer”
cactttccagtctctgcgcggtacgttctttctatcatcccg

SEQ ID NO: 66 - DNA, 41 bp, Artificial Sequence
  source: /note=“Description of Artificial Sequence: Synthetic primer”
ctctcatggaatggctaaagacaagcacggtgagcgtgaac

SEQ ID NO: 67 - DNA, 17 bp, Artificial Sequence
  source: /note=“Description of Artificial Sequence: Synthetic primer”
gctttgagggggcctga

SEQ ID NO: 68 - DNA, 22 bp, Artificial Sequence
  source: /note=“Description of Artificial Sequence: Synthetic primer”
accaatcttgtcacctctgact

SEQ ID NO: 69 - DNA, 2877 bp, Unknown
  source: /note=“Description of Unknown: Bacteriophage T7 sequence”
tcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacagggctcgcaagtctcgccgtatcagccttgagttcatgcttgagcaagccaataagtttgctaaccataaggccatctggttcccttacaacatggactggcgcggtcgtgtttacgccgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgagaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggctcacggtgttactcgcagtgtgactaagcgttcagtcatgacgctggcttacgggtccaaagagttcggcttccgtcaacaagtgctggaagataccattcagccagctattgattccggcaagggtccgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcc

SEQ ID NO: 70 - DNA, 2655 bp, Unknown
  source: /note=“Description of Unknown: Bacteriophage T7 sequence”
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaataa

SEQ ID NO: 71 - DNA, 2759 bp, Unknown
  source: /note=“Description of Unknown: Bacteriophage SP6 sequence”
gcgctcaattaagttttctagtaccgcatgaggatacaagatgcaagatttacacgctatccagcttcaattagaagaagagatgtttaatggtggcattcgtcgcttcgaagcagatcaacaacgccagattgcagcaggtagcgagagcgacacagcatggaaccgccgcctgttgtcagaacttattgcacctatggctgaaggcattcaggcttataaagaagagtacgaaggtaagaaaggtcgtgcacctcgcgcattggctttcttacaatgtgtagaaaatgaagttgcagcatacatcactatgaaagttgttatggatatgctgaatacggatgctacccttcaggctattgcaatgagtgtagcagaacgcattgaagaccaagtgcgcttttctaagctagaaggtcacgccgctaaatactttgagaaggttaagaagtcactcaaggctagccgtactaagtcatatcgtcacgctcataacgtagctgtagttgctgaaaaatcagttgcagaaaaggacgcggactttgaccgttgggaggcgtggccaaaagaaactcaattgcagattggtactaccttgcttgaaatcttagaaggtagcgttttctataatggtgaacctgtatttatgcgtgctatgcgcacttatggcggaaagactatttactacttacaaacttctgaaagtgtaggccagtggattagcgcattcaaagagcacgtagcgcaattaagcccagcttatgccccttgcgtaatccctcctcgtccttggagaactccatttaatggagggttccatactgagaaggtagctagccgtatccgtcttgtaaaaggtaaccgtgagcatgtacgcaagttgactcaaaagcaaatgccaaaggtttataaggctatcaacgcattacaaaatacacaatggcaaatcaacaaggatgtattagcagttattgaagaagtaatccgcttagaccttggttatggtgtaccttccttcaagccactgattgacaaggagaacaagccagctaacccggtacctgttgaattccaacacctgcgcggtcgtgaactgaaagagatgctatcacctgagcagtggcaacaattcattaactggaaaggcgaatgcgcgcgcctatataccgcagaaactaagcgcggttcaaagtccgccgccgttgttcgcatggtaggacaggcccgtaaatatagcgcctttgaatccatttacttcgtgtacgcaatggatagccgcagccgtgtctatgtgcaatctagcacgctctctccgcagtctaacgacttaggtaaggcattactccgctttaccgagggacgccctgtgaatggcgtagaagcgcttaaatggttctgcatcaatggtgctaacctttggggatgggacaagaaaacttttgatgtgcgcgtgtctaacgtattagatgaggaattccaagatatgtgtcgagacatcgccgcagaccctctcacattcacccaatgggctaaagctgatgcaccttatgaattcctcgcttggtgctttgagtatgctcaataccttgatttggtggatgaaggaagggccgacgaattccgcactcacctaccagtacatcaggacgggtcttgttcaggcattcagcactatagtgctatgcttcgcgacgaagtaggggccaaagctgttaacctgaaaccctccgatgcaccgcaggatatctatggggcggtggcgcaagtggttatcaagaagaatgcgctatatatggatgcggacgatgcaaccacgtttacttctggtagcgtcacgctgtccggtacagaactgcgagcaatggctagcgcatgggatagtattggtattacccgtagcttaaccaaaaagcccgtgatgaccttgccatatggttctactcgcttaacttgccgtgaatctgtgattgattacatcgtagacttagaggaaaaagaggcgcagaaggcagtagcagaagggcggacggcaaacaaggtacatccttttgaagacgatcgtcaagattacttgactccgggcgcagcttacaactacatgacggcactaatctggccttctatttctgaagtagttaaggcaccgatagtagctatgaagatgatacgccagcttgcacgctttgcagcgaaacgtaatgaaggcctgatgtacaccctgcctactggcttcatcttagaacagaagatcatggcaaccgagatgctacgcgtgcgtacctgtctgatgggtgatatcaagatgtcccttcaggttgaaacggatatcgtagatgaagccgctatgatgggagcagcagcacctaatttcgtacacggtcatgacgcaagtcaccttatccttaccgtatgtgaattggtagacaagggcgtaactagtatcgctgtaatccacgactcttttggtactcatgcagacaacaccctcactcttagagtggcacttaaagggcagatggttgcaatgtatattgatggtaatgcgcttcagaaactactggaggagcatgaagtgcgctggatggttgatacaggtatcgaagtacctgagcaaggggagttcgaccttaacgaaatcatggattctgaatacgtatttgcctaatagaacaataaatatacaggtcagccttcgggctggccttttcttttaactattacctgtaacatttaattaacaagtccaacgtgttggacac

SEQ ID NO: 72 - DNA, 18 bp, Artificial Sequence
  source: /note=“Description of Artificial Sequence: Synthetic oligonucleotide”
taatacgactcactatag

SEQ ID NO: 73 - DNA, 18 bp, Artificial Sequence
  source: /note=“Description of Artificial Sequence: Synthetic oligonucleotide”
atttaggtgacactatag